(19) United States
(12) Patent Application Publication
(10) Pub. No.: US 2022/0233298 A1
(43) Pub. Date: Jul. 28, 2022

(54) A BIOCOMPATIBLE MEMBRANE COMPOSITE

(71) Applicants: W. L. Gore & Associates, Inc., Newark, DE (US); Viacyte, Inc., San Diego, CA (US)

(72) Inventors: Bahman Aghdasi, Newark, DE (US); Timothy M. Bruhn, Newark, DE (US); Kevin D'Amour, San Diego, CA (US); Edward Gunzel, Newark, DE (US); Laura Martinson, San Diego, CA (US); Scott A. Ritrovato, Newark, DE (US); Greg Rusch, Newark, DE (US); Michael Scott, San Diego, CA (US); Lauren R. Zambotti, Newark, DE (US); Qiang (John) Zhang, San Diego, CA (US); Christopher Folk, San Diego, CA (US); Craig McGreevy, San Diego, CA (US); Joseph Kakkassery, San Diego, CA (US)

(21) Appl. No.: 17/595,911

(22) PCT Filed: May 30, 2020
(86) PCT No.: PCT/US2020/035450

§ 371 Date: Nov. 29, 2021

Related U.S. Application Data
(60) Provisional application No. 62/855,707, filed on May 31, 2019.

(51) Int. Cl. A61F 2/02; A61L 27/16; A61L 27/34; A61L 27/38; A61L 27/54

(52) U.S. Cl. CPC A61F 2/022; A61L 27/16; A61L 27/34; A61L 2420/08; A61L 27/54; A61L 2400/08; A61L 2400/18; A61L 27/3804

(57) ABSTRACT

A biocompatible membrane composite that can provide an environment that is able to mitigate or tailor the foreign body response is provided. The membrane composite contains a mitigation layer and a vascularization layer. A reinforcing component may optionally be included to provide support to and prevent distortion of the biocompatible membrane composite in vivo. The mitigation layer may be bonded (e.g., point bonded or welded) or adhered (intimately or discretely) to an implantable device and/or cell system. The biocompatible membrane composite may be used as a surface layer for implantable devices or cell systems that require vascularization for function but need protection from the host's immune response, such as the formation of foreign body giant cells. The biocompatible membrane composite may partially or fully cover the exterior of an implantable device or cell system. The mitigation layer is positioned between the implantable device or bioactive scaffold and the vascularization layer.

### FIELD

The present invention relates generally to the field of implantable devices and, in particular, to a biocompatible membrane composite and uses thereof.

### BACKGROUND

Biological therapies are increasingly viable methods for treating peripheral artery disease, aneurysm, heart disease, Alzheimer's and Parkinson's diseases, autism, blindness, diabetes, and other pathologies.

With respect to biological therapies in general, cells, viruses, viral vectors, bacteria, proteins, antibodies, and other bioactive moieties may be introduced into a patient by surgical or interventional methods that place the bioactive moiety into a tissue bed of a patient. Often the bioactive moieties are first placed in a device that is then inserted into a patient. Alternatively, the device may be inserted into a patient first with the bioactive moiety added later.

The implantation of external devices (e.g., cell encapsulation devices, sensors, and/or monitors for measuring physical parameters and/or analytes in the body) triggers an immune response in which foreign body giant cells form and at least partially encapsulate the implanted device. The device may be formed of one or more biocompatible membranes or other biocompatible materials that permit the passage of nutrients or other therapeutically useful substances through but prevent the passage of the cells therethrough. The presence of foreign body giant cells at or near the cell impermeable interface makes it difficult, if not impossible for blood vessels to form in close proximity to this surface, thereby restricting access to the oxygen, nutrients, analytes or other signaling across the device interface needed for adequate device function.

Thus, there remains a need in the art for a material that can be utilized in or that can provide an environment that is able to mitigate or tailor the foreign body response such that sufficient vascularization occurs at or near the surface of a cell impermeable interface, thereby permitting the implanted, encapsulated cells to survive and secrete a therapeutically useful substance and that permits the implanted device access to analytes and physical parameters for measurement.

### SUMMARY

In one Aspect, (“Aspect 1”), a biocompatible membrane composite includes a first layer having first solid features with a first solid feature spacing, where a majority of the first solid feature spacing is less than about 50 microns, and a second layer having second solid features with a second solid feature spacing, where a majority of the second solid feature spacing is greater than about 50 microns.

According to another Aspect, (“Aspect 2”) further to Aspect 1, the first layer includes a majority of a representative minor axis from about 3 microns to about 20 microns.

According to another Aspect, (“Aspect 3”) further to Aspect 1 or Aspect 2, the second layer has a first pore size greater than about 9 microns in effective diameter.

According to another Aspect, (“Aspect 4”) further to any one of Aspects 1 to 3, the first layer has a first thickness less than about 200 microns.

According to another Aspect, (“Aspect 5”) further to any one of Aspects 1 to 4, the first layer has a second pore size from about 1 micron to about 9 microns in effective diameter.

According to another Aspect, (“Aspect 6”) further to Aspect 5, the solid features of at least one of the first layer and the second layer are connected by fibrils and the fibrils are deformable.

According to another Aspect, (“Aspect 7”) further to any one of Aspects 1 to 5, the second layer has a second thickness from about 30 microns to about 200 microns.

According to another Aspect, (“Aspect 8”) any one of Aspects 1 to 6, at least one of the first layer and the second layer includes a polymer selected from an expanded polytetrafluoroethylene (ePTFE) membrane, a fluorinated ethylene propylene (FEP) membrane and a modified ePTFE membrane.

According to another Aspect, (“Aspect 9”) further to any one of Aspects 1 to 8, the biocompatible membrane composite has thereon a surface coating that includes one or more members selected from antimicrobial agents, antibodies, pharmaceuticals, and biologically active molecules.

According to another Aspect, (“Aspect 10”) further to any one of Aspects 1 to 9, at least one of the first layer and the second layer is an expanded polytetrafluoroethylene membrane.

According to another Aspect, (“Aspect 11”) further to any one of Aspects 1 to 10, the second layer is a spunbound non-woven polyester material.

According to another Aspect, (“Aspect 12”) further to any one of Aspects 1-10, including a reinforcing layer.

According to another Aspect, (“Aspect 13”) further to Aspect 12, the reinforcing layer is a woven or non-woven textile.

According to another Aspect, (“Aspect 14”) further to any one of Aspects 1 to 13, the solid features of the first layer includes a representative minor axis, a representative major axis, and a solid feature depth, and where a majority of at least two of the representative minor axis, the representative major axis, and the solid feature depth are greater than about 5 microns.

In another Aspect, (“Aspect 15”), further to any one of Aspects 1 to 14, including a first layer having a first pore size from about 1 micron to about 9 microns in effective diameter, a first thickness less than about 200 microns, and first solid features having a majority of a first solid feature spacing less than about 50 microns, where a majority of the first solid features have a first representative minor axis from about 3 microns to about 20 microns and a second layer.

According to another Aspect, (“Aspect 16”) further to any one of Aspects 1 to 15, the second layer has a pore size greater than about 9 microns in effective diameter.

According to another Aspect, (“Aspect 17”) further to any one of Aspects 1 to 16, the second layer includes second solid features with a majority of a second solid feature spacing greater than about 50 microns.

According to another Aspect, (“Aspect 18”) further to any one of Aspects 15 to 17, the second layer has a second thickness from about 30 microns to about 200 microns.

According to another Aspect, (“Aspect 19”) further to any one of Aspects 15 to 18, the first solid features of the first layer each include a majority of a first representative major axis and a first solid feature depth, where a majority of at least two of the first representative minor axis, the first representative major axis, and the first solid feature depth are greater than about 5 microns.

According to another Aspect, (“Aspect 20”) further to any one of Aspects 15 to 19, the solid features are connected by fibrils and the fibrils are deformable.

According to another Aspect, (“Aspect 21”) further to any one of Aspects 15 to 20, the second layer includes second solid features and a majority of the second solid features has a second representative minor axis that is less than about 40 microns.

According to another Aspect, (“Aspect 22”) further to any one of Aspects 15 to 21, the second layer includes a second representative major axis and a second solid feature depth, and wherein a majority of at least two of the second representative minor axis, the second representative major axis, and the second solid feature depth is greater than about 5 microns.

According to another Aspect, (“Aspect 23”) further to any one of Aspects 15 to 22, where at least one of the first layer and the second layer is a polymer selected from an expanded polytetrafluoroethylene (ePTFE) membrane, a fluorinated ethylene propylene (FEP) membrane and a modified ePTFE membrane.

According to another Aspect, (“Aspect 24”) further to any one of Aspects 15 to 23, the second layer is a spunbound non-woven polyester material.

According to another Aspect, (“Aspect 25”) further to any one of Aspects 15 to 24, at least one of the first layer and the second layer includes a polymer, fluoropolymer membranes, non-fluoropolymer membranes, a woven biocompatible textile, a non-woven biocompatible textile, woven or non-woven collections of fibers or yarns, fibrous matrices, and combinations thereof.

According to another Aspect, (“Aspect 26”) further to any one of Aspects 15 to 25, the first solid features of the first layer include a member selected from a thermoplastic polymer, polyurethanes, silicones, rubbers, epoxies and combinations thereof.

According to another Aspect, (“Aspect 27”) further to any one of Aspects 15 to 26, including a reinforcing component.

According to another Aspect, (“Aspect 28”) further to Aspect 27, the reinforcing component is a woven or non-woven textile.

According to another Aspect, (“Aspect 29”) further to any one of Aspects 15 to 28, the biocompatible membrane composite has thereon a surface coating that includes one or more members selected from antimicrobial agents, antibodies, pharmaceuticals, and biologically active molecules.

According to another Aspect, (“Aspect 30”) further to any one of Aspects 15 to 29, the biocompatible membrane composite has a hydrophilic coating thereon.

According to another Aspect, (“Aspect 31”) further to any one of Aspects 15 to 30, the first layer includes bondable solid features where the bondable solid features are bonded to an implantable device or implantable cell system.

According to another Aspect, (“Aspect 32”) further to Aspect 31, the implantable device is a scaffold.

According to another Aspect, (“Aspect 33”) further to Aspect 32, the scaffold is a cell culture matrix.

According to another Aspect, (“Aspect 34”) further to Aspect 32, the scaffold is an explant.

According to another Aspect, (“Aspect 35”) further to Aspect 31, the first solid features are at least partially bonded to a cell system.

According to another Aspect, (“Aspect 36”) further to Aspect 35, the cell system is a cell container.

According to another Aspect, (“Aspect 37”) further to Aspect 31, the implantable device is a sensor.

According to another Aspect, (“Aspect 38”) further to Aspect 31, the cell system is a bioactive scaffold.

According to another Aspect (“Aspect 39”) further to any of the preceding Aspects, a method for lowering blood glucose levels in a mammal includes transplanting a cell encapsulated device including a biocompatible membrane composite of any of the previous claims, where cells encapsulated therein include a population of PDX1-positive pancreatic endoderm cells, and where the pancreatic endoderm cells mature into insulin secreting cells, thereby lowering blood glucose.

According to another Aspect (“Aspect 40”) further to any of the preceding Aspects, the PDX1-positive pancreatic endoderm cells include a mixture of cells further including endocrine and/or endocrine precursor cells, where the endocrine and/or endocrine precursor cells express chromogranin A (CHGA).

According to another Aspect (“Aspect 41”) further to any of the preceding Aspects, a method for lowering blood glucose levels in a mammal transplanting a cell encapsulation device as in claim 1, where cells encapsulated therein include a population of PDX1-positive pancreatic endoderm cells, and where the pancreatic endoderm cells mature into insulin secreting cells, thereby lowering blood glucose.

According to another Aspect (“Aspect 42”) further to any of the preceding Aspects, the PDX1-positive pancreatic endoderm cells include a mixture of cells further including endocrine and/or endocrine precursor cells, where the endocrine and/or endocrine precursor cells express chromogranin A (CHGA).

According to another Aspect (“Aspect 43”) further to any of the preceding Aspects, a method for lowering blood glucose levels in a mammal includes transplanting a cell encapsulation device including at least one sensor and a biocompatible membrane composite that at least partially covers the sensor where the biocompatible membrane composite includes a first layer having first solid features with a majority of a first solid feature spacing less than about 50 microns and a second layer having second solid features with a majority of a second solid feature spacing greater than about 50 microns, where the first layer is positioned between the sensor and the second layer, where at least a portion of the bonded features are intimately bonded to the first layer, and a cell population including PDX1-positive pancreatic endoderm cells, and where the pancreatic endoderm cells mature into insulin secreting cells, thereby lowering blood glucose.

According to another Aspect (“Aspect 44”) further to any of the preceding Aspects, the PDX1-positive pancreatic endoderm cells include a mixture of cells further including endocrine and/or endocrine precursor cells, where the endocrine and/or endocrine precursor cells express chromogranin A (CHGA).

According to another Aspect (“Aspect 45”) further to any of the preceding Aspects, a method for lowering blood glucose levels in a mammal includes transplanting at least one sensor and a biocompatible membrane composite that at least partially covers the sensor where the biocompatible membrane composite includes a first layer having first solid features with a majority of a first solid feature spacing less than about 50 microns and a second layer having second solid features with a majority of a second solid feature spacing greater than about 50 microns, where the first layer is positioned between the sensor and the second layer, where at least a portion of the bonded features are intimately bonded to the first layer, and a cell population including PDX1-positive pancreatic endoderm cells, and where the pancreatic endoderm cells mature into insulin secreting cells, thereby lowering blood glucose.

According to another Aspect (“Aspect 46”) further to any of the preceding Aspects, the PDX1-positive pancreatic endoderm cells include a mixture of cells further including endocrine and/or endocrine precursor cells, where the endocrine and/or endocrine precursor cells express chromogranin A (CHGA).

According to another Aspect (“Aspect 47”) further to any of the preceding Aspects, an encapsulated in vitro PDX1-positive pancreatic endoderm cells include a mixture of cell sub-populations including at least a pancreatic progenitor population co-expressing PDX-1/NKX6.1.

According to another Aspect (“Aspect 48”) further to any of the preceding Aspects, an encapsulated in vitro PDX1-positive pancreatic endoderm cells includes a mixture of cell sub-populations including at least a pancreatic progenitor population co-expressing PDX-1/NKX6.1 and a pancreatic endocrine and/or endocrine precursor population expressing PDX-1/NKX6.1 and CHGA.

According to another Aspect (“Aspect 49”) further to any of the preceding Aspects, at least 30% of the population includes pancreatic progenitor population co-expressing PDX-1/NKX6.1.

According to another Aspect (“Aspect 50”) further to any of the preceding Aspects, at least 40% of the population includes pancreatic progenitor population co-expressing PDX-1/NKX6.1.

According to another Aspect (“Aspect 51”) further to any of the preceding Aspects, at least 50% of the population includes pancreatic progenitor population co-expressing PDX-1/NKX6.1.

According to another Aspect (“Aspect 52”) further to any of the preceding Aspects, at least 20% of the population endocrine and/or endocrine precursor population express PDX-1/NKX6.1/CHGA.

According to another Aspect (“Aspect 53”) further to any of the preceding Aspects, at least 30% of the population endocrine and/or endocrine precursor population express PDX-1/NKX6.1/CHGA.

According to another Aspect (“Aspect 54”) further to any of the preceding Aspects, at least 40% of the population endocrine and/or endocrine precursor population express PDX-1/NKX6.1/CHGA.

According to another Aspect (“Aspect 55”) further to any of the preceding Aspects, the pancreatic progenitor cells and/or endocrine or endocrine precursor cells are capable of maturing into insulin secreting cells in vivo.

According to another Aspect (“Aspect 56”) further to any of the preceding Aspects, a method for producing insulin in vivo includes transplanting a cell encapsulated device including a biocompatible membrane composite of any of the previous claims and a population of PDX-1 pancreatic endoderm cells mature into insulin secreting cells, where the insulin secreting cells secrete insulin in response to glucose stimulation.

According to another Aspect (“Aspect 57”) further to any of the preceding Aspects, the PDX1-positive pancreatic endoderm cells include a mixture of cells further including endocrine and/or endocrine precursor cells, where the endocrine and/or endocrine precursor cells express chromogranin A (CHGA).

According to another Aspect (“Aspect 58”) further to any of the preceding Aspects, at least about 30% of the population are endocrine and/or endocrine precursor population expressing PDX-1/NKX6.1/CHGA.

According to another Aspect (“Aspect 59”) further to any of the preceding Aspects, an in vitro human PDX1-positive pancreatic endoderm cell culture includes a mixture of PDX-1 positive pancreatic endoderm cells and at least a transforming growth factor beta (TGF-beta) receptor kinase inhibitor.

According to another Aspect (“Aspect 60”) further to any of the preceding Aspects, further including a bone morphogenetic protein (BMP) inhibitor.

According to another Aspect (“Aspect 61”) further to any of the preceding Aspects, the TGF-beta receptor kinase inhibitor is TGF-beta receptor type 1 kinase inhibitor.

According to another Aspect (“Aspect 62”) further to any of the preceding Aspects, the TGF-beta receptor kinase inhibitor is ALK5i.

According to another Aspect (“Aspect 63”) further to any of the preceding Aspects, the BMP inhibitor is noggin.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a schematic illustration depicting the determination of solid feature spacing where three neighboring solid features represent the corners of a triangle whose circumcircle has an interior devoid of additional solid features and the solid feature spacing is the straight distance between two of the solid features forming the triangle in accordance with embodiments described herein;

FIG. 1B is a schematic illustration depicting the determination of non-neighboring solid features where the solid features form the corners of a triangle whose circumcircle contains at least one additional solid feature in accordance with embodiments described herein;

FIG. 2 is a scanning electron micrograph of the spacing (white lines) between solid features (white shapes) in an ePTFE membrane in accordance with embodiments described herein;

FIG. 3A is a schematic illustration depicting the method to determine the major axis and the minor axis of a solid feature in accordance with embodiments described herein;

FIG. 3B is a schematic illustration depicting the depth of a solid feature in accordance with embodiments described herein;

FIG. 4 is a schematic illustration of the effective diameter of a pore in accordance with embodiments described herein;

FIG. 5 is a scanning electron micrograph (SEM) showing a pore size according to embodiments described herein;

FIG. 6A is a schematic illustration of a cross-sectional view of an implantable device that may be at least partially be covered by a biocompatible membrane composite in accordance with embodiments herein;

FIG. 6B is a schematic illustration of a bioactive scaffold that may be at least partially covered by a biocompatible membrane composite in accordance with embodiments described herein;

FIG. 7 is a schematic illustration of a biocompatible membrane composite in accordance with embodiments described herein;

FIG. 8 is a schematic illustration of another biocompatible membrane composite in accordance with embodiments described herein;

FIG. 9 is a schematic illustration of yet another biocompatible membrane composite in accordance with embodiments described herein;

FIG. 10 is a scanning electron micrograph (SEM) of the top surface of the ePTFE mitigation layer of Example 1 in accordance with embodiments described herein;

FIG. 11 is a scanning electron micrograph (SEM) of the top surface of a vascularization layer formed of a non-woven polyester utilized in Example 1 in accordance with embodiments described herein; and

FIG. 12 is an exploded view of the configuration of materials and fixtures utilized in Example 1 in accordance with embodiments described herein;

FIG. 13 is a representative SEM image of the second ePTFE layer of Constructs A, B, and C of Example 2 having thereon FEP in accordance with embodiments described herein;

FIG. 14 is a representative SEM image of the node and fibril structure of the second ePTFE membrane in Construct A of Example 2 in accordance with embodiments described herein;

FIG. 15 is a representative SEM image of the node and fibril structure of the second ePTFE membrane in Construct B of Example 2 in accordance with embodiments described herein;

FIG. 16 is a representative SEM image of the node and fibril structure of the second ePTFE membrane in Construct C of Example 2 in accordance with embodiments described herein;

FIG. 17 is an SEM image of the cross-section of the biocompatible membrane composite of Construct A of Example 2 in accordance with embodiments described herein;

FIG. 18 is an SEM image of the cross-section of the biocompatible membrane composite of Construct B of Example 2 in accordance with embodiments described herein; and

FIG. 19 is an SEM image of the cross-section of the biocompatible membrane composite of Construct C of Example 2 in accordance with embodiments described herein.

### DETAILED DESCRIPTION

Persons skilled in the art will readily appreciate that various aspects of the present disclosure can be realized by any number of methods and apparatus configured to perform the intended functions. It should also be noted that the accompanying figures referred to herein are not necessarily drawn to scale, and may be exaggerated to illustrate various aspects of the present disclosure, and in that regard, the figures should not be construed as limiting. Directional references such as “up,” “down,” “top,” “left,” “right,” “front,” and “back,” among others are intended to refer to the orientation as illustrated and described in the figure (or figures) to which the components and directions are referencing. It is to be appreciated that the terms “biocompatible membrane composite” and “membrane composite” are used interchangeably herein. It is to be noted that all ranges described herein are exemplary in nature and include any and all values in between. In addition, all references cited herein are incorporated by reference in their entireties.

The present disclosure is directed to a biocompatible membrane composite that can provide an environment that is able to mitigate or tailor the foreign body response. The biocompatible membrane composite contains a first layer and a second layer. Each layer is distinct, serving a unique function that aids in mitigating the formation of foreign body giant cells on a cell impermeable layer of an implantable device or bioactive entity (e.g., bioactive scaffold). In certain embodiments, the first layer functions as a mitigation layer and the second layer that functions as a vascularization layer. Herein, the term “first layer” is used interchangeably with “mitigation layer” and the term “second layer” is used interchangeably with “vascularization layer” for ease of convenience. The mitigation layer is positioned between the implantable device or bioactive entity and the vascularization layer. In at least one embodiment, the mitigation layer includes solid features (e.g., nodes) that are inherently present in the membrane forming the mitigation layer. A reinforcing component may optionally be positioned on either side of the biocompatible membrane composite (i.e., external to) or within the biocompatible membrane composite (i.e., internal to) to provide support to and prevent distortion of the biocompatible membrane composite. The mitigation layer may be to be bonded (e.g., point bonded or welded) to the implantable device and/or bioactive entity. In some embodiments, the mitigation layer and the vascularization layer may be intimately bonded or otherwise connected to each other to form a composite layer having an open/open structure. As used herein, the terms “intimate bond” and “intimately bonded” refer to layers of the biocompatible membrane composite or to solid features within the biocompatible membrane composite that are not readily separable or detachable at any point on their surface. It is to be appreciated that the term “about” as used herein denotes +/−10% of the designated unit of measure.

In at least one embodiment, the mitigation layer and the vascularization layer are bonded together by one or more biocompatible adhesive to form the biocompatible membrane composite. The adhesive may be applied to the surface of one or both of the mitigation layer and the vascularization layer in a manner to create a discrete or intimate bond between the layers. As used herein, the phrases “discrete bond” or “discretely bonded” are meant to include bonding in intentional patterns of points and/or lines around a continuous perimeter of a defined region. Non-limiting examples of suitable biocompatible adhesives include fluorinated ethylene propylene (FEP), a polycarbonate urethane, a thermoplastic fluoropolymer comprised of TFE and PAVE, EFEP (ethylene fluorinated ethylene propylene), PEBAX (a polyether amide), PVDF (poly vinylidene fluoride), CarbOSil® (absilicone polycarbonate urethane), Elasthane™ (a polyether urethane), PurSil® (a silicone polyether urethane), polyethylene, high density polyethylene (HDPE), ethylene chlorotetrafluoroethylene (ECTFE), perfluoroalkoxy (PFA), polypropylene, polyethylene terephthalate (PET), and combinations thereof.

In some embodiments, the biocompatible membrane composites described herein may be utilized as a bio-interface for implantable sensors that are used to detect molecules produced in the body (such as glucose or other biologically active molecules) or molecules that are produced outside the body (such as molecules from ingested food). In another embodiment, the biocompatible membrane composites may be used as a biocompatible cover for implantable devices that provide or require molecules, signals, or activity within the body to elicit their function, such as, for example, pacemakers. The implantable device may be used to measure physical parameters of a body, such as, for example, blood pressure. Herein, the term “implantable device” is used to encompass any implantable sensor or implantable device. In other embodiments, the biocompatible membrane composites may be used as a surface layer or as an encompassing cover for implantable devices that require vascularization for function but need protection from the host's immune response, such as, but not limited to, the formation of foreign body giant cells. The implantable device may contain thereon a third layer (i.e., cell impermeable layer). The cell impermeable layer serves as a microporous, immune isolation barrier, is impervious to vascular ingrowth, and prevents cellular contact from a host. In another embodiment, the biocompatible membrane composites may be used in conjunction with tissues, cell scaffolds, or cell encapsulation devices. Some examples include, but are not limited to, explants, two-dimensional (2D) and three-dimensional (3D) cell culture systems or cell containers. The collective term “cell system” is utilized herein to describe any biological entity that may be used in conjunction with the biocompatible membrane composite.

Elements of implantable devices that could benefit from the function of the biocompatible membrane composites include, but are not limited to, switches, sensors, bolometers, biosensors, chemical sensors, inertial sensors, acoustic sensors, microphones, microspeakers, pressure sensors, resonators, ultrasonic resonators, temperature sensors, vibration sensors, microengines, actuators, thermal actuators, bimorph and unimorph actuators (e.g., piezo and thermo), electrical rotating micromachines, microgears, micropumps, microtransmiitors, microengines, optical micro-electro-mechanical systems (MEMS), micromirrors, optical switches, and bio-micro-electro-mechanical systems (MEMS).

The interface of the biocompatible membrane composite with the implantable device is the mitigation layer, which is sufficiently porous to permit growth of vascular tissue into the mitigation layer. Thus, in some instances, the mitigation layer acts as an initial vascularization layer. The mitigation layer creates a suitable environment to minimize or even prevent the formation of contiguous layer of foreign body giant cells on or near a surface of the implantable device, while allowing blood vessels to access the surface of the implantable device. Herein, layers that have openings large enough to allow vascular ingrowth may be referred to as “open” layers. Blood vessels, which are the source of analytes and nutrients for the implantable device, need to form at a distance from the implantable device so that the signals are easily detected and transmitted. Non-limiting examples of the signal include glucose, oxygen, a growth factor, or any analyte that is in need of sensing or monitoring.

The mitigation layer is characterized at least in part by the inclusion of a plurality of solid features that have a solid feature spacing. “Solid features” as used herein may be defined as three dimensional components within the mitigation layer that are generally immovable and resistant to deformation when exposed to environmental forces such as, but not limited to, cell movement (e.g., cellular migration and ingrowth, host vascularization/endothelial blood vessel formation). The solid features in the mitigation layer may be formed of thermoplastic polymers, polyurethanes, silicones, rubbers, epoxies, and combinations thereof.

In embodiments where the mitigation layer has a node and fibril microstructure (e.g. formed from a fibrillated polymer), the nodes are the solid features and the fibrils are not solid features. Indeed, in some embodiments, the fibrils may be removed, leaving only the nodes in the mitigation layer. In embodiments where the nodes within the mitigation layer are the solid features, those nodes which are intimately bonded to the device or sensor interface and are herein referred to as “bonded solid features”. “Non-bonded solid features” are those solid features within the mitigation layer that are not bonded (intimately bonded or otherwise) to the device or sensor interface. In one embodiment, the mitigation layer is formed of an expanded polytetrafluoroethylene (ePTFE) membrane having a node and fibril microstructure.

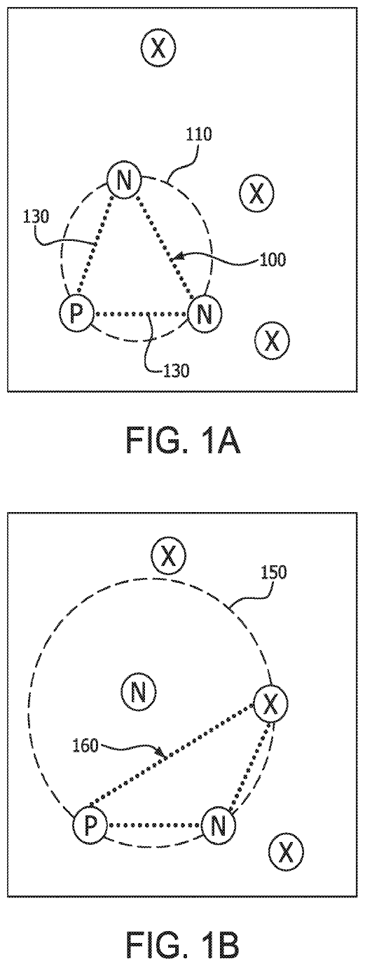

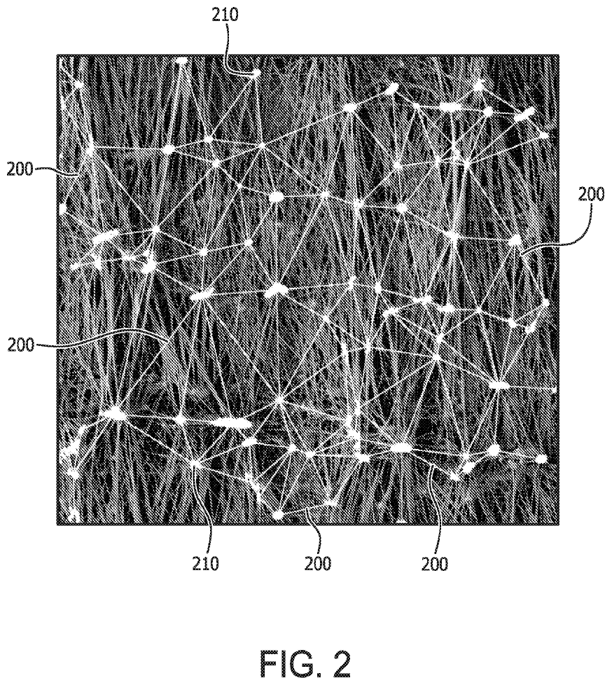

The majority of the solid feature spacing of the solid features adjacent to the implantable device or cell system is less than about 50 microns, less than about 40 microns, less than about 30 microns, less than about 20 microns, or less than about 10 microns. As used herein, the term “majority” is meant to describe an amount over half (i.e., greater than 50%) of the measured values for the parameter being measured. In some embodiments, the majority of the solid feature spacing may range from about 5 microns to about 45 microns, from about 10 microns to about 40 microns, from about 10 microns to about 35 microns, or from about 15 microns to about 35 microns. The phrase “solid feature spacing” is defined herein as the straight-line distance between two neighboring solid features. In this disclosure, solid features are considered neighboring if their centroids represent the corners of a triangle whose circumcircle has an empty interior. As shown pictorially in FIG. 1A, the designated solid feature (P) is connected to neighboring solid features (N) to form a triangle 100 where the circumcircle 110 contains no solid features within. Solid features (X) designate the solid features that are not neighboring solid features. Thus, in the instance depicted in FIG. 1A, the solid feature spacing 130 is the straight distance between the designated solid features (P), (N). In contrast, the circumcircle 150 shown in FIG. 1B drawn from the triangle 160 contains therein a solid feature (N), and as such, cannot be utilized to determine the solid feature spacing in the mitigation layer (or the vascularization layer). FIG. 2 is a scanning electron micrograph depicting measured distances, e.g., the white lines 200 between the solid features 210 (white shapes) in a mitigation layer formed of an expanded polytetrafluoroethylene membrane.

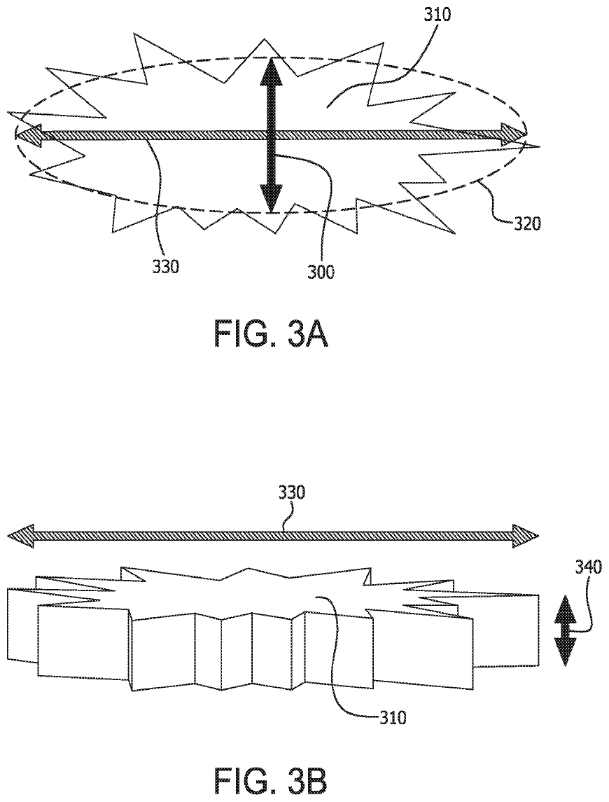

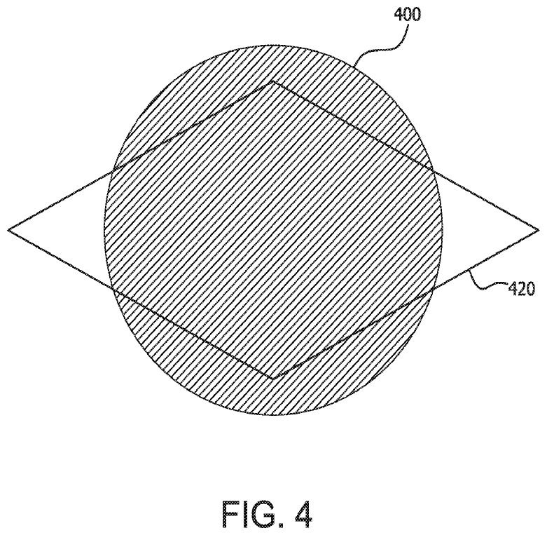

The solid features also include a representative minor axis, a representative major axis, and a solid feature depth. The representative minor axis of a solid feature is defined herein as the length of the minor axis of an ellipse fit to the solid feature where the ellipse has the same area, orientation, and centroid as the solid feature. The representative major axis of a solid feature is defined herein as the length of the major axis of an ellipse fit to the solid feature where the ellipse has the same area, orientation, and centroid as the solid feature. The major axis is greater than or equal to the minor axis in length. The minor and major axes of an ellipse 320 to fit the solid feature 310 is shown pictorially in FIG. 3A. The representative minor axis of the solid feature 310 is depicted by arrow 300, and the representative major axis of the solid feature 310 is depicted by arrow 330. A majority of the solid features has a minor axis that ranges in size from about 3 microns to about 20 microns, from about 3 microns to about 15 microns, or from about 3 microns to about 10 microns. The solid feature depth is the length of the projection of the solid feature in the axis perpendicular to the surface of the layer (e.g., mitigation layer or vascularization layer). The solid feature depth of the solid feature 310 is shown pictorially in FIG. 3B. The depth of the solid feature 310 is depicted by line 340. In at least one embodiment, the depth of the solid features is equal to or less than the thickness of the mitigation layer. In at least one embodiment, a majority of at least two of the representative minor axis, representative major axis, and solid feature depth is greater than 5 microns.

In embodiments where the solid features are interconnected by fibrils or fibers, the boundary connecting the solid features creates a pore. It is necessary that these pores are open enough to allow rapid cellular ingrowth and vascularization and not create a resistance to mass transport of oxygen and nutrients. The pore effective diameter is measured by quantitative image analysis (QIA) and performed on a scanning electron micrograph (SEM) image. The term “effective diameter” of a pore is defined as the diameter of a circle that has an area equal to the measured area of the surface pore. This relationship is defined by the following equation:

$$Effective Diameter = 2 \times \sqrt{\frac{Area}{π}} .$$

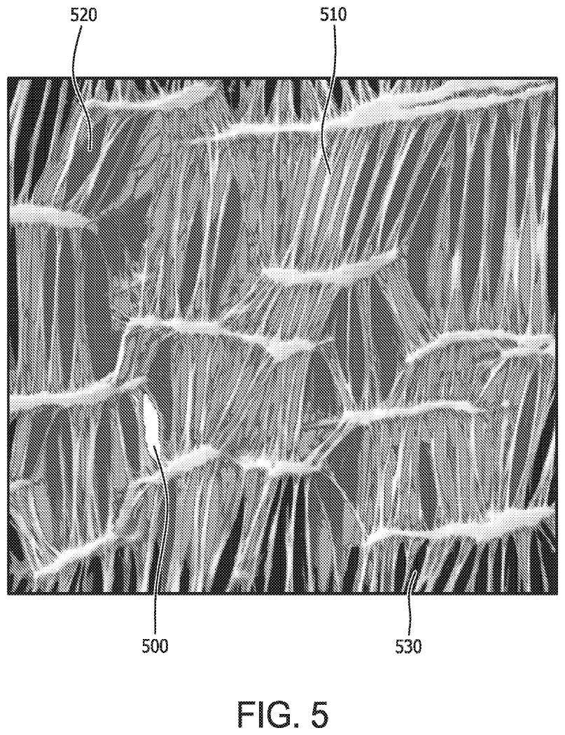

Turning to FIG. 4, the effective diameter is the diameter of the circle 400 and the surface pore is designated by reference numeral 420. The total pore area of a surface is the sum of the area of all pores at that surface. The pore size of a layer is the effective diameter of the pore that defines the point where roughly half the total pore area consists of pores with diameters smaller than the pore size and half the total pore area consists of pores with diameters greater than or equal to the pore size. FIG. 5 illustrates a pore size 500 (white in color), pores smaller in size 510 (shown in light grey), and pores larger in size 520 (shown in dark grey). Pores that intersect with the edge of the image 530 were excluded from analysis and are shown in black.

The pore size of the mitigation layer may range from about 1 micron to about 9 microns in effective diameter, from about 3 microns in effective diameter to about 9 microns in effective diameter, or from about 4 micron in effective diameter to about 9 microns in effective diameter as measured by quantitative image analysis (QIA) performed on a scanning electron micrograph (SEM) image. The mitigation layer has a thickness that is less than about 200 microns, less than about 290 microns, less than about 280 microns, less than about 270 microns, less than about 260 microns, less than about 200 microns, less than about 190 microns, less than about 180 microns, less than about 170 microns, less than about 160 microns, less than about 150 microns, less than about 140 microns, less than about 130 microns, less than about 120 microns, less than about 110 microns, less than about 100 microns, less than about 90 microns, less than about 80 microns, less than about 70 microns, or less than about 60 microns, less than 50 about microns, less than about 40 microns, less than about 30 microns, less than about 20 microns, or less than about 10 microns. The thickness of the mitigation layer may range from about 60 microns to about 200 microns, from about 60 microns to about 170 microns, from about 60 to about 150 microns, from about 60 microns to about 125 microns, from about 60 microns to about 100 microns, from about 3 microns to about 60 microns, from about 10 microns to about 50 microns, from about 10 microns to about 40 microns, or from about 15 microns to about 35 microns. In some embodiments, the mitigation layer has a porosity greater than about 60%. In other embodiments, the mitigation layer has a porosity greater than about 70%, greater than about 80%, greater than about 90%, or greater than about 95%. In some embodiments, the porosity may be about 98% or about 99%. The porosity of the mitigation layer may range from about 60% to about 98%, from about 70% to about 98%, or from about 80% to about 98%.

The anchoring of the implantable device and ingrowth of vascular tissue through the biocompatible membrane composite up to the surface of the device is further facilitated by the second layer (i.e., vascularization layer). The vascularization layer is an “open” layer that permits additional vascular penetration from the host and also permits rapid anchoring and attachment of the biocompatible membrane composite within the tissue of the host. Additionally, the vascularization layer provides a porous matrix to harbor the growth of a sufficient quantity of additional, new blood vessels, such as to the implantable device or the cell system. In embodiments where the vascularization layer does not meet the same criteria of the mitigation layer the mitigation layer and vascularization layer are considered as separate and distinct layers. The vascularization layer is designed such that there are solid features to enable host integration and attachment. These solid features have increased spacing and pore sizes therebetween compared to the solid features of the mitigation layer to facilitate a more rapid ingrowth of tissue into the layer.

In some embodiments, the majority of the solid feature spacing of the solid features in the vascularization layer is greater than about 50 microns, greater than about 60 microns, greater than about 70 microns, or greater than about 80 microns. A majority of the solid features in the vascularization layer has a solid feature spacing that range from about 50 microns to about 90 microns, from about 60 microns to about 90 microns, or from about 70 microns to about 90 microns. The pore size and overall thickness of the vascularization layer is sufficient to provide space to harbor the necessary quantities of additional blood vessels to provide nutrients and oxygen to cells. A pore size of the vascularization layer may be greater than about 9 microns in effective diameter, greater than about 25 microns in effective diameter, greater than about 50 microns in effective diameter, greater than about 75 microns in effective diameter, greater than about 100 microns in effective diameter, greater than about 125 microns in effective diameter, greater than about 150 microns in effective diameter, greater than about 175 microns in effective diameter, or greater than about 200 microns in effective diameter as measured by QIA performed on an SEM image. In some embodiments, the pore size of the vascularization layer may range from about 9 microns in effective diameter to about 200 microns in effective diameter, from about 9 microns in effective diameter to about 50 microns in effective diameter, from about 15 microns in effective diameter to about 50 microns in effective diameter from about 25 microns in effective diameter to about 50 microns in effective diameter, from about 50 microns in effective diameter to about 200 microns in effective diameter, from about 75 microns in effective diameter to about 175 microns in effective diameter as measured by QIA performed on an SEM image.

Additionally, the vascularization layer may have a thickness that is greater than about 30 microns, greater than about 50 microns, greater than about 75 microns, greater than about 100 microns, greater than about 125 microns, greater than about 150 microns, or greater than about 200 microns. In addition, the thickness of the vascularization layer may range from about 30 microns to about 300 microns, from about 30 microns to about 200 microns, from about 30 microns to about 100 microns, from about 100 microns to about 200 microns, or from about 100 microns to about 150 microns. In addition, a majority of the solid features in the vascularization layer has a representative minor axis that is less than about 40 microns, less than about 30 microns, less than about 20 microns, less than about 10 microns, less than about 5 microns, or less than about 3 microns. In some embodiments, the representative minor axis may range in size from about 3 microns to about 40 microns, from about 3 microns to about 30 microns, from about 3 microns to about 20 microns, from about 3 microns to about 10 microns, or from about 20 microns to about 40 microns. The solid features in the vascularization layer also have a major axis that greater in length than the minor axis and may effectively be unlimited in length, such as a continuous fiber of a non-woven. The solid features in the vascularization layer have a depth that is less than or equal to the total thickness of the vascularization layer.

An optional reinforcing component may be included to provide mechanical support to the biocompatible membrane composite to minimize distortion in vivo. This additional optional reinforcing component provides a stiffness to the biocompatible membrane composite that is greater than the biocompatible membrane composite itself. This optional reinforcing component could be continuous in nature or it may be present in discrete regions on the biocompatible membrane composite, e.g., patterned across the entire surface of the biocompatible membrane composite or located in specific locations such as around the perimeter of the biocompatible membrane composite. Non-limiting patterns suitable for the surface of the membrane composite include dots, straight lines, angled lines, curved lines, dotted lines, grids, etc. Patterns forming the reinforcing component may be used singly or in combination. In addition, the reinforcing component may be temporary in nature (e.g., formed of a bioabsorbable material) or may be permanent in nature (e.g., a polyethylene terephthalate (PET) mesh or Nitinol). A final determination of the component stiffness depends not only on the stiffness of a single reinforcing component, but also on the location and restraint of the reinforcing component in the final device form.

In at least one embodiment, the reinforcing component may be provided on the outer surface of the vascularization layer to strengthen the biocompatible membrane composite against environmental forces. In this orientation, the reinforcing component has a pore size sufficient to permit vascular ingrowth, and is therefore is considered an “open” layer. Materials useful as the reinforcing component include materials that are significantly stiffer than the biocompatible membrane composite. Such materials include, but are not limited to, open mesh biomaterial textiles, woven textiles, non-woven textiles (e.g., collections of fibers or yarns), and fibrous matrices, either alone or in combination.

In some embodiments, the mitigation layer and vascularization layer may be bonded together by one or more biocompatible adhesive to form the biocompatible membrane composite. The adhesive may be applied to the surface of one or both of the mitigation layer and vascularization layer in a manner to create a discrete or intimate bond between the layers. Non-limiting examples of suitable biocompatible adhesives include fluorinated ethylene propylene (FEP), a polycarbonate urethane, a thermoplastic fluoropolymer comprised of TFE and PAVE, EFEP (ethylene fluorinated ethylene propylene), PEBAX (a polyether amide), PVDF (poly vinylidene fluoride), CarbOSil® (absilicone polycarbonate urethane), Elasthane™ (a polyether urethane), PurSil® (a silicone polyether urethane), polyethylene, high density polyethylene (HDPE), ethylene chlorotetrafluoroethylene (ECTFE), perfluoroalkoxy (PFA), polypropylene, polyethylene terephthalate (PET), and combinations thereof.

In some embodiments, at least one of the mitigation layer and the vascularization layer may be formed of a polymer membrane or woven or non-woven collections of fibers or yarns, or fibrous matrices, either alone or in combination. Non-limiting examples of polymers that may be used include, but are not limited to, alginate; cellulose acetate; polyalkylene glycols such as polyethylene glycol and polypropylene glycol; panvinyl polymers such as polyvinyl alcohol; chitosan; polyacrylates such as polyhydroxyethylmethacrylate; agarose; hydrolyzed polyacrylonitrile; polyacrylonitrile copolymers; polyvinyl acrylates such as polyethylene-co-acrylic acid, polyalkylenes such as polypropylene, polyethylene; polyvinylidene fluoride; fluorinated ethylene propylene (FEP); perfluoroalkoxy alkane (PFA); polyester sulfone (PES); polyurethanes; polyesters; and copolymers and combinations thereof. In some embodiments, the vascularization layer may be a spunbound, non-woven polyester or an expanded polytetrafluoroethylene (ePTFE) membrane.

In some embodiments at least one of the mitigation layer, the vascularization layer, or the reinforcing component is formed of a non-woven fabric. There are numerous types of non-woven fabrics, each of which may vary in tightness of the weave and the thickness of the sheet. The filament cross-section may be trilobal. The non-woven fabric may be a bonded fabric, a formed fabric, or an engineered fabric that is manufactured by processes other than weaving or knitting. In some embodiments, the non-woven fabric is a porous, textile-like material, usually in a flat sheet form, and composed primarily or entirely of fibers, such as staple fibers assembled in a web, sheet, or batt. The structure of the non-woven fabric is based on the arrangement of, for example, staple fibers that are typically randomly arranged. In addition, non-woven fabrics can be created by a variety of techniques known in the textile industry. Various methods may create carded, wet laid, melt blown, spunbonded, or air laid non-woven materials. Methods and substrates are described, for example, in U.S. Patent Publication No. 2010/0151575 to Colter, et al. In one embodiment, the non-woven fabric is polytetrafluoroethylene (PTFE). In another embodiment, the non-woven fabric is a spunbound polyester. The density of the non-woven fabric may be varied depending upon the processing conditions. In one embodiment, the non-woven fabric is a spunbound polyester with a basic weight from about 10 to about 20 g/m²a nominal thickness from about 75 to about 150 microns, and a fiber diameter from about 20 to about 40 microns. The filament cross-section is trilobal. The filament cross-section is trilobal. In some embodiments, the non-woven fabrics are bioabsorbable.

In some embodiments, the polymer(s) forming the polymer membrane of the mitigation layer and/or vascularization layer is a fibrillatable polymer. Fibrillatable, as defined herein, refers to the ability to introduce fibrils to a polymer membrane including, but not limited to, converting portions of the solid features into fibrils. For example, the fibrils are the solid elements that span the gaps between the solid features. Fibrils are generally not resistant to deformation upon exposure to environmental forces, and are therefore deformable. The majority of deformable fibrils in the mitigation layer and/or vascularization layer may have a diameter less than about 2 microns, less than about 1 micron, less than about 0.75 microns, less than about 0.50 microns, or less than about 0.25 microns. In some embodiments, the fibrils may have a diameter from about 0.25 microns to about 2 microns, from about 0.5 microns to about 2 microns, or from about 0.75 microns to about 2 microns.

In some embodiments, the solid features of one or both of the mitigation layer and the vascularization layer may be formed by microlithography, micro-molding, machining, selectively depositing, or printing (or otherwise laying down) a polymer (e.g., thermoplastic) onto a mitigation layer or a vascularization layer to form at least a part of a solid feature. Any conventional printing technique such as transfer coating, screen printing, gravure printing, ink-jet printing, patterned imbibing, and knife coating may be utilized to place the thermoplastic polymer onto the mitigation layer and/or vascularization layer. Optionally, the pattern may be printed onto a liner and applied to the mitigation layer, vascularization layer, or an implantable device.

Materials used to form the solid features include, but are not limited to, thermoplastics, polyurethane, polypropylene, silicones, rubbers, epoxies, polyethylene, polyether amide, polyetheretherketone, polyphenylsulfone, polysulfone, silicone polycarbonate urethane, polyether urethane, polycarbonate urethane, silicone polyether urethane, polyester, polyester terephthalate, melt-processable fluoropolymers, such as, for example, fluorinated ethylene propylene (FEP), tetrafluoroethylene-(perfluoroalkyl) vinyl ether (PFA), an alternating copolymer of ethylene and tetrafluoroethylene (ETFE), a terpolymer of tetrafluoroethylene (TFE), hexafluoropropylene (HFP) and vinylidene fluoride (THV), polyvinylidene fluoride (PVDF), and combinations thereof. In some embodiments, polytetrafluoroethylene may be used to form the pattern features. In further embodiments, the solid features may be separately formed and adhered to the surface of the vascularization layer or surface of the implantable device (not illustrated).

Non-limiting examples of fibrillatable polymers that may be used to form one or more of the mitigation layer, and the vascularization layer, and optional cell impermeable layer include, but are not limited to, tetrafluoroethylene (TFE) polymers such as polytetrafluoroethylene (PTFE), expanded PTFE (ePTFE), modified PTFE, TFE copolymers, polyvinylidene fluoride (PVDF), poly (p-xylylene) (ePPX) as taught in U.S. Patent Publication No. 2016/0032069 to Sbriglia, porous ultra-high molecular weight polyethylene (eUHMWPE) as taught in U.S. Pat. No. 9,926,416 to Sbriglia, porous ethylene tetrafluoroethylene (eETFE) as taught in U.S. Pat. No. 9,932,429 to Sbriglia, and porous vinylidene fluoride-co-tetrafluoroethylene or trifluoroethylene [VDF-co-(TFE or TrFE)] polymers as taught in U.S. Pat. No. 9,441,088 to Sbriglia and combinations thereof.

In some embodiments, the fibrillatable polymer is a fluoropolymer membrane such as expanded polytetrafluoroethylene (ePTFE) membrane. Expanded polytetrafluoroethylene (ePTFE) (and other fibrillated polymers) has a node and fibril microstructure where the nodes are interconnected by the fibrils and the pores are the space located between the nodes and fibrils throughout the membrane. As used herein, the term “node” is meant to denote a solid feature consisting of largely of polymer material. When deformable fibrils are present, these nodes reside at the junction of multiple fibrils. In some embodiments the fibrils may be removed from the membrane, such as, for example, by plasma etching. In at least one embodiment, an expanded polytetrafluoroethylene membrane is used in one or more of the mitigation layer, the vascularization layer and the optional cell impermeable layer. Expanded polytetrafluoroethylene membranes such as, but not limited to, those prepared in accordance with the methods described in U.S. Pat. No. 3,953,566 to Gore, U.S. Pat. No. 7,306,729 to Bacino et al., U.S. Pat. No. 5,476,589 to Bacino, WO 94/13469 to Bacino, U.S. Pat. No. 5,814,405 to Branca et al. or U.S. Pat. No. 5,183,545 to Branca et al. may be used herein.

In some embodiments, one or more of the mitigation layer and the vascularization layer may be formed of a fluoropolymer membrane, such as, but not limited to, an expanded polytetrafluoroethylene (ePTFE) membrane, a modified ePTFE membrane, a tetrafluoroethylene (TFE) copolymer membrane, a polyvinylidene fluoride (PVDF) membrane, or a fluorinated ethylene propylene (FEP) membrane. In further embodiments, the vascularization layer may include biocompatible textiles, including wovens and non-wovens (e.g., a spunbound non-woven, melt blown fibrous materials, electrospun nanofibers, etc.), non-fluoropolymer membranes such as polyvinylidene difluoride (PVDF), nanofibers, polysulfones, polyethersulfones, polyarlysulfones, polyether ether ketone (PEEK), polyethylenes, polypropylenes, and polyimides. In some embodiments, the vascularization layer is a spunbound, non-woven polyester or an expanded polytetrafluoroethylene (ePTFE) membrane.

In some embodiments, it may be desirable for one or more of the vascularization layer and reinforcing component to be non-permeant (e.g., biodegradable). In such instances, a biodegradable material may be used to form the vascularization layer and/or the reinforcing component. Suitable examples of biodegradable materials include, but are not limited to, polyglycolide:trimethylene carbonate (PGA:TMC), polyalphahydroxy acid such as polylactic acid, polyglycolic acid, poly (glycolide), and poly(lactide-co-caprolactone), poly(caprolactone), poly(carbonates), poly(dioxanone), poly (hydroxybutyrates), poly(hydroxyvalerates), poly (hydroxybutyrates-co-valerates), expanded polyparaxylylene (ePLLA), such as is taught in U.S. Patent Publication No. 2016/0032069 to Sbriglia, and copolymers and blends thereof. Alternatively, the vascularization layer may be coated with a bio-absorbable material or a bio-absorbable material may be incorporated into or onto the vascularization layer in the form of a powder. Coated materials may promote infection site reduction, vascularization, and favorable type 1 collagen deposition.

The biocompatible membrane composite may have at least partially thereon a surface coating, such as a Zwitterion non-fouling coating, a hydrophilic coating, or a CBAV/Fleparin coating (commercially available from W. L. Gore & Associates, Inc.). The surface coating may also or alternatively contain antimicrobial agents, antibodies (e.g., anti-CD 47 antibodies (anti-fibrotic)), pharmaceuticals, biologically active molecules (e.g., stimulators of vascularization such as FGF, VEGF, endoglin, PDGF, angiopoetins, and integrins; Anti-fibrotic such as TGFb inhibitors, sirolimus, CSF1R inhibitors, anti-inflammatory/immune modulators such as CXCL12, and corticosteroids), and combinations thereof.

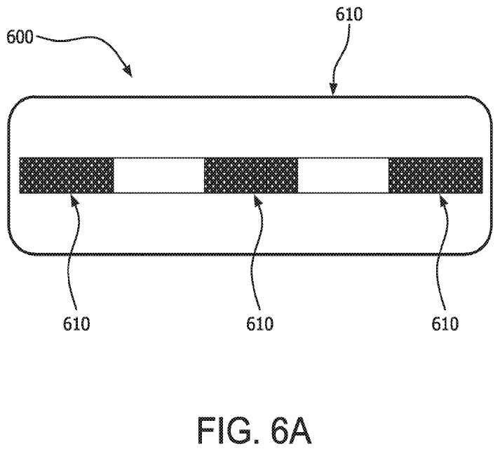

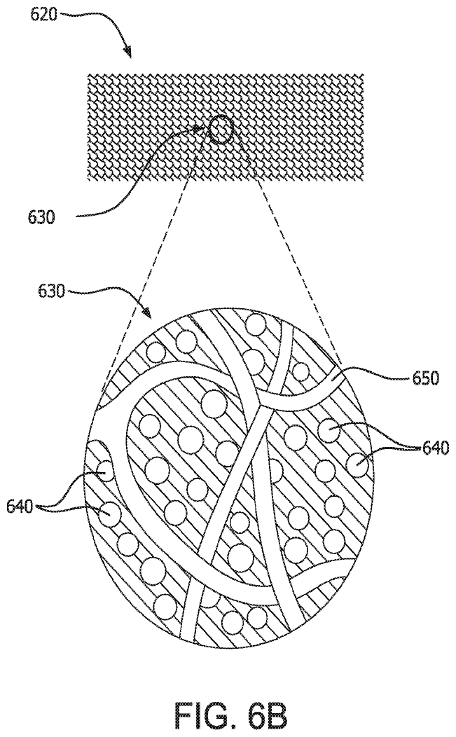

Turning to FIG. 6A, in at least one embodiment, the biocompatible membrane composite may be used in combination with an implantable device 600. In particular, the biocompatible membrane composite (not shown) may partially or fully cover the enclosure 605. Enclosure 605 may be a pouch or container for carrying components 610 of a sensor, pacemaker, or electrical lead, or it may be the implantable device itself. In another embodiment depicted in FIG. 6B, the biocompatible membrane composite (not shown) may partially or fully cover the exterior of the cell system 620 and/or a portion or all of the structural elements 650. Section 630 is magnified to show individual structural elements 650 of the cell system and cells 640 growing with cell system 620.

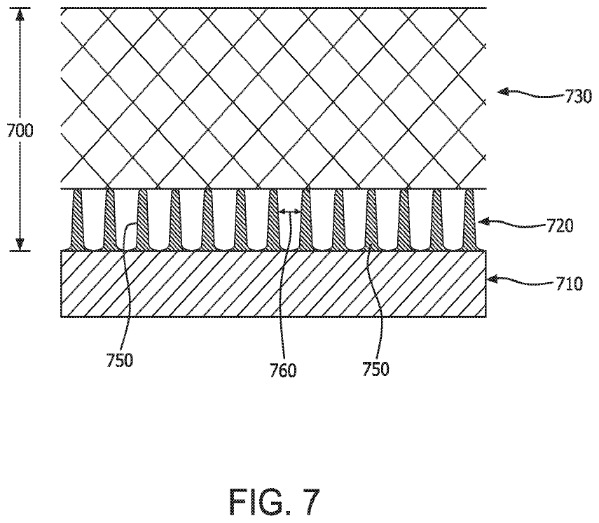

A biocompatible membrane composite 700 is depicted in FIG. 7. As illustrated in FIG. 7, the biocompatible membrane composite 700 includes a mitigation layer (i.e., first layer) 720 and a vascularization layer (i.e., second layer) 730. The biocompatible membrane composite 700 may be utilized to at least partially cover, encompass, or surround an implantable device 710. In the depicted embodiment, solid features 750 are attached to the surface of an implantable device 710 to form the mitigation layer 720. “Attached” as used herein is mean to include intimately attached or discretely attached. In some embodiments, the solid features 750 do not penetrate into the vascularization layer 730. The solid features 750 are depicted in FIG. 7 as being essentially the same height and width and extending between the implantable device 710 and the vascularization layer 730, although it is to be appreciated this is an example and the solid features 750 may vary in height and/or width. The distance between solid features 750 is the solid feature spacing 760.

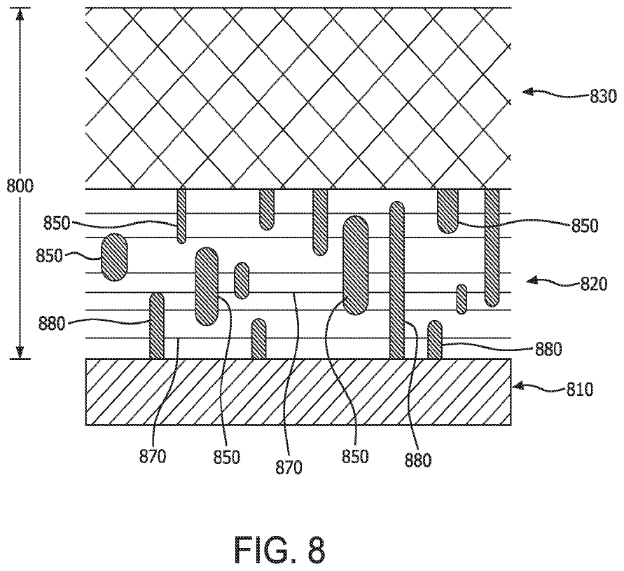

FIG. 8 is another biocompatible composite. As illustrated in FIG. 8, the biocompatible membrane composite 800 includes a mitigation layer 820 and a vascularization layer 830. In the depicted embodiment, the solid features 850 are nodes that differ in height and width, and may or may not extend the distance between the implantable device 810 and the vascularization layer 830. The solid features 850 are connected by fibrils 870. In FIG. 8, the majority of the solid feature depth is less than the total thickness of the mitigation layer 820. Bondable solid features 880 may be attached to the surface of the implantable device 810.

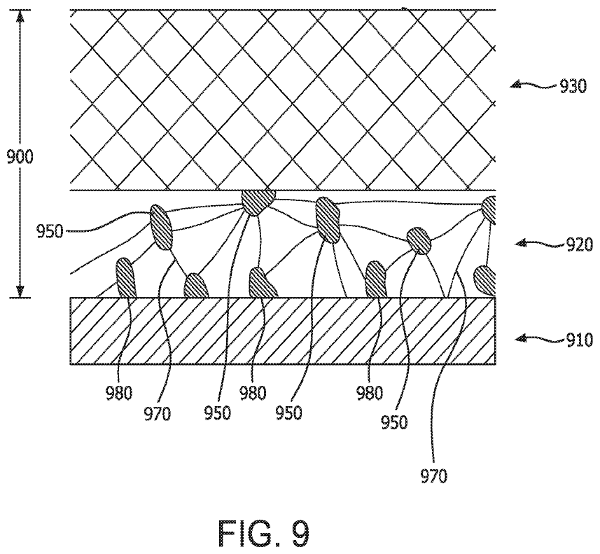

Turning to FIG. 9, a biocompatible membrane composite 900 is shown. The biocompatible membrane composite 900 includes a mitigation layer 920 and a vascularization layer 930. The biocompatible membrane composite 900 may at least partially cover or encompass the implantable device 910. In this embodiment, solid features within the mitigation layer 920 are nodes formed of an expanded polytetrafluoroethylene membrane. The nodes 950 are interconnected by fibrils 970. Nodes 950, 980 are positioned within the mitigation layer 920. Bondable solid features or nodes 980, however, are not only within the mitigation layer 920, but also are in contact with, and may be intimately bonded to, the implantable device 910.

It is to be appreciated that in each of the embodiments described in FIGS. 7-9, a cell system may replace the implantable device and such embodiments are considered to be within the purview of the invention.

### Test Methods

### Porosity

The porosity of a layer is defined herein as the proportion of layer volume consisting of pore space compared to the total volume of the layer. The porosity is calculated by comparing the bulk density of a porous construct consisting of solid fraction and void fraction to the density of the solid fraction using the following equation:

$$Porosity = ( 1 - \frac{D e n s i t {y}_{Bulk}}{D e n s i t {y}_{Solid Fraction}} ) \times 100 \% .$$

### Mass/Area

Samples were cut (either by hand, laser, or die) to a known geometry. The dimensions of the sample were measured or verified and the area was calculated in m². The sample was then weighed in grams on a calibrated scale. The mass in grams was divided by the area in m² to calculate the mass per area in g/m².

### Thickness

The thickness of the layers in the biocompatible membrane composites were measured by quantitative image analysis (QIA) of cross-sectional SEM images. Cross-sectional SEM images were generated by fixing membranes to an adhesive, cutting the film by hand using a liquid-nitrogen-cooled razor blade, and then standing the adhesive backed film on end such that the cross-section was vertical. The sample was then sputter coated using an Emitech K550X sputter coater (commercially available from Quorum Technologies Ltd, UK) and platinum target. The sample was then imaged using a FEI Quanta 400 scanning electron microscope from Thermo Scientific.

Layers within the cross-section SEM images were then measured for thickness using ImageJ 1.51 h from the National Institutes of Health (NIH). The image scale was set per the scale provided by the SEM. The layer of interest was isolated and cropped using the free-hand tool. A number of at least ten equally spaced lines were then drawn in the direction of the layer thickness. The lengths of all lines were measured and averaged to define the layer thickness.

### Stiffness

A stiffness test was performed based on ASTM D790-17 Standard test method for flexural properties of unreinforced and reinforced plastics and electrical insulating material. This method was used to determine the stiffness for biocompatible membrane composite layers and/or the final device.

Procedure B of the ASTM method was followed and includes greater than 5% strain and type 1 crosshead position for deflection. The dimensions of the fixture were adjusted to have a span of 16 mm and a radius of support and nosepiece of 1.6 mm. The test parameters used were a deflection of 3.14 mm and a test speed of 96.8 mm/min. In cases where the sample width differed from the standard 1 cm, the force was normalized to a 1 cm sample width by the linear ratio.

The load was reported in N/cm at maximum deflection.

### SEM Sample Preparation

SEM samples were prepared by first fixing the membrane composite or membrane composite layer(s) of an adhesive for handling, with the side opposite the side intended for imaging facing the adhesive. The film was then cut to provide an approximately 3 mm×3 mm area for imaging. The sample was then sputter coated using an Emitech K550X sputter coater and platinum target. Images were then taken using a FEI Quanta 400 scanning electron microscope from Thermo Scientific at a magnificent and resolution that allowed visualization of a sufficient number of features for robust analysis while ensuring each feature's minimum dimension was at least five pixels in length.

### Solid Feature Spacing

Solid feature was determined by analyzing SEM images in ImageJ 1.51 h from the National Institute of Health (NIH). The image scale was set based on the scale provided by the SEM image. Features were identified and isolated through a combination of thresholding based on size/shading and/or manual identification. In instances where the structure consists of a continuous structure, such as a nonwoven or etched surface, as opposed to a structure with discrete solid features, solid features are defined as the portion of the structure surrounding voids the their corresponding spacing extending from one side of the void to the opposing side. After isolating the features, a Delaunay Triangulation was performed to identify neighboring features. Triangulations whose circumcircle extended beyond the edge of the image were disregarded from the analysis. Lines were drawn between the nearest edges of neighboring features and measured for length to define spacing between neighboring features (see, e.g., FIG. 1A).

The median of all measured solid feature spacings marks the value that is less than or equal to half of the measured solid feature spacings and greater than or equal to half of the measured solid feature spacings. Therefore, if the measured median is above or below some value, the majority of measurements is similarly above or below the value. As such, the median is used as summary statistic to represent the majority of solid feature spacings.

### Measurement of Representative Minor Axis and Representative Major Axis

The representative minor axis was measured by analyzing SEM images of membrane surfaces in ImageJ 1.51 h from the NIH. The image scale was set based on the scale provided by the SEM image. Features were identified and isolated through a combination of thresholding based on size/shading and/or manual identification. After isolating the features, the built in particle analysis capabilities were leveraged to determine the major and minor axis of the representative ellipse. The minor axis of this ellipse is the representative minor axis of the measured feature. The major axis of this ellipse is the representative major axis of the measured feature. The median of all measured minor axes marks the value that is less than or equal to half of the measured minor axes and greater than or equal to half of the measured minor axes. Similarly, the median of all measured major axes marks the value that is less than or equal to half of the measured major axes and greater than or equal to half of the measured major axes. In both cases, if the measured median is above or below some value, the majority of measurements is similarly above or below the value. As such, the median is used as summary statistic to represent the majority of solid feature representative minor axes and representative major axes.

### Solid Feature Depth

Solid feature depth was determined by using quantitative image analysis (QIA) of SEM images of membrane cross-sections. Cross-sectional SEM images were generated by fixing films to an adhesive, cutting the film by hand using a liquid-nitrogen-cooled razor blade, and then standing the adhesive backed film on end such that the cross-section was vertical. The sample was then sputter coated using an Emitech K550X sputter coater (commercially available from Quorum Technologies Ltd, UK) and platinum target. The sample was then imaged using a FEI Quanta 400 scanning electron microscope from Thermo Scientific.

Features within the cross-section SEM images were then measured for depth using ImageJ 1.51 h from the National Institutes of Health (NIH). The image scale was set per the scale provided by the SEM. Features were identified and isolated through a combination of thresholding based on size/shading and/or manual identification. After isolating features, built in particle analysis capabilities were leveraged to calculate the Feret diameter and angle formed by the axis defined by the Feret diameter axis and horizontal plane for each solid feature. The Feret diameter is the furthest distance between any two points on a feature's boundary in the plane of the SEM image. The Feret diameter axis is the line defined by these two points. The projection of the Feret diameter of each solid feature in the direction of the layer thickness was calculated per the equation.

$${Projection}_{T h i c k n e s s} = sin θ * Len g t {h}_{L o n gest Axis} .$$

The projection of the longest axis in the direction of the layer thickness is the solid feature depth of the measured feature. The median of all measured solid feature depths marks the value that is less than or equal to half of the measured solid feature depths and greater than or equal to half of the measured solid feature depths. Therefore, if the measured median is above or below some value, the majority of measurements is similarly above or below the value As such, the median is used as summary statistic to represent the majority of solid feature depths.

### Pore Size

The pore size was measured by analyzing SEM images of membrane surfaces in ImageJ 1.51 h from the NIH. The image scale was set based on the scale provided by the SEM image. Pores were identified and isolated through a combination of thresholding based on size/shading and/or manual identification. After isolating the pores, the built in particle analysis capabilities were leveraged to determine the area of each pore. The measured pore area was converted to an “effective diameter” per the below equation:

$$Effective Diameter = 2 \times \sqrt{\frac{Area}{π}}$$

The pore areas were summed to define the total area of the surface defined by pores. This is the total pore area of the surface. The pore size of a layer is the effective diameter of the pore that defines the point where roughly half the total pore area consists of pores with diameters smaller than the pore size and roughly half the total pore area consists of pores with diameters greater than or equal to the pore size.

### In Vitro Production of Human PDX1-Positive Pancreatic Endoderm and Endocrine Cells

The directed differentiation methods herein for pluripotent stem cells, for example, hES and iPS cells, can be described into at least four or five or six or seven stages, depending on end-stage cell culture or cell population desired (e.g. PDX1-positive pancreatic endoderm cell population (or PEC), or endocrine precursor cell population, or endocrine cell population, or immature beta cell population or mature endocrine cell population).

Stage 1 is the production of definitive endoderm from pluripotent stem cells and takes about 2 to 5 days, preferably 2 or 3 days. Pluripotent stem cells are suspended in media comprising RPMI, a TGFβ superfamily member growth factor, such as Activin A, Activin B, GDF-8 or GDF-11 (100 ng/mL), a Wnt family member or Wnt pathway activator, such as Wnt3a (25 ng/mL), and alternatively a rho-kinase or ROCK inhibitor, such as Y-27632 (10 μM) to enhance growth, and/or survival and/or proliferation, and/or cell-cell adhesion. After about 24 hours, the media is exchanged for media comprising RPMI with serum, such as 0.2% FBS, and a TGFβ superfamily member growth factor, such as Activin A, Activin B, GDF-8 or GDF-11 (100 ng/mL), and alternatively a rho-kinase or ROCK inhibitor for another 24 (day 1) to 48 hours (day 2).

Alternatively, after about 24 hours in a medium comprising Activin/Wnt3a, the cells are cultured during the subsequent 24 hours in a medium comprising Activin alone (i.e., the medium does not include Wnt3a). Importantly, production of definitive endoderm requires cell culture conditions low in serum content and thereby low in insulin or insulin-like growth factor content. See McLean et al. (2007) Stem Cells 25: 29-38. McLean et al. also show that contacting hES cells with insulin in concentrations as little as 0.2 μg/mL at Stage 1 can be detrimental to the production of definitive endoderm. Still others skilled in the art have modified the Stage 1 differentiation of pluripotent cells to definitive endoderm substantially as described here and in D'Amour et al. (2005), for example, at least, Agarwal et al., Efficient Differentiation of Functional Hepatocytes from Human Embryonic Stem Cells, Stem Cells (2008) 26:1117-1127; Borowiak et al., Small Molecules Efficiently Direct Endodermal Differentiation of Mouse and Human Embryonic Stem Cells, (2009) Cell Stem Cell 4:348-358; Brunner et al., Distinct DNA methylation patterns characterize differentiated human embryonic stem cells and developing human fetal liver, (2009) Genome Res. 19:1044-1056, Rezania et al. Reversal of Diabetes with Insulin-producing Cells Derived In Vitro from Human Pluripotent Stem Cells (2014) Nat Biotech 32(11): 1121-1133 (GDF8 & GSK3beta inhibitor, e.g. CHIR99021); and Pagliuca et al. (2014) Generation of Function Human Pancreatic B-cell In Vitro, Cell 159: 428-439 (Activin A & CHIR) Proper differentiation, specification, characterization and identification of definitive are necessary in order to derive other endoderm-lineage cells. Definitive endoderm cells at this stage co-express SOX17 and HNF3I3 (FOXA2) and do not appreciably express at least HNF4alpha, HNF6, PDX1, SOX6, PROX1, PTF1A, CPA, cMYC, NKX6.1, NGN3, PAX3, ARX, NKX2.2, INS, GSC, GHRL, SST, or PP. The absence of HNF4alpha expression in definitive endoderm is supported and described in detail in at least Duncan et al. (1994), Expression of transcription factor HNF-4 in the extraembryonic endoderm, gut, and nephrogenic tissue of the developing mouse embryo: HNF-4 is a marker for primary endoderm in the implanting blastocyst,” Proc. Natl. Acad. Sci, 91:7598-7602 and Si-Tayeb et al. (2010), Highly Efficient Generation of Human Hepatocyte-Like cells from Induced Pluripotent Stem Cells,” Hepatology 51:297-305.

Stage 2 takes the definitive endoderm cell culture from Stage 1 and produces foregut endoderm or PDX1-negative foregut endoderm by incubating the suspension cultures with RPMI with low serum levels, such as 0.2% FBS, in a 1:1000 dilution of ITS, 25 ng KGF (or FGF7), and alternatively a ROCK inhibitor for 24 hours (day 2 to day 3). After 24 hours (day 3 to day 4), the media is exchanged for the same media minus a TGFβ inhibitor, but alternatively still a ROCK inhibitor to enhance growth, survival and proliferation of the cells, for another 24 (day 4 to day 5) to 48 hours (day 6). A critical step for proper specification of foregut endoderm is removal of TGFβ family growth factors. Hence, a TGFβ inhibitor can be added to Stage 2 cell cultures, such as 2.5 μM TGFβ inhibitor no. 4 or 5 μM SB431542, a specific inhibitor of activin receptor-like kinase (ALK), which is a TGFβ type I receptor. Foregut endoderm or PDX1-negative foregut endoderm cells produced from Stage 2 co-express SOX17, HNF1p and HNF4alpha and do not appreciably co-express at leasHNF3β (FOXA2), nor HNF6, PDX1, SOX6, PROX1, PTF1A, CPA, cMYC, NKX6.1, NGN3, PAX3, ARX, NKX2.2, INS, GSC, GHRL, SST, or PP, which are hallmark of definitive endoderm, PDX1-positive pancreatic endoderm or pancreatic progenitor cells or endocrine progenitor/precursors as well as typically poly hormonal type cells.

Stage 3 (days 5-8) for PEC production takes the foregut endoderm cell culture from Stage 2 and produces a PDX1-positive foregut endoderm cell by DMEM or RPMI in 1% B27, 0.25 μM KAAD cyclopamine, a retinoid, such as 0.2 retinoic acid (RA) or a retinoic acid analog such as 3 nM of TTNPB (or CTT3, which is the combination of KAAD cyclopamine and TTNPB), and 50 ng/mL of Noggin for about 24 (day 7) to 48 hours (day 8). Specifically, Applicants have used DMEM-high glucose since about 2003 and all patent and non-patent disclosures as of that time employed DMEM-high glucose, even if not mentioned as “DMEM-high glucose” and the like. This is, in part, because manufacturers such as Gibco did not name their DMEM as such, e.g. DMEM (Cat. No 11960) and Knockout DMEM (Cat. No 10829). It is noteworthy, that as of the filing date of this application, Gibco offers more DMEM products but still does not put “high glucose” in certain of their DMEM products that contain high glucose e.g. Knockout DMEM (Cat. No. 10829-018). Thus, it can be assumed that in each instance DMEM is described, it is meant DMEM with high glucose and this was apparent by others doing research and development in this field. Again, a ROCK inhibitor or rho-kinase inhibitor such as Y-27632 can be used to enhance growth, survival, proliferation and promote cell-cell adhesion. Additional agents and factors include but are not limited to ascorbic acid (e.g. Vitamin C), BMP inhibitor (e.g. Noggin, LDN, Chordin), SHH inhibitor (e.g. SANT, cyclopamine, HIP1); and/or PKC activator (e.g. PdBu, TBP, ILV) or any combination thereof. Alternatively, Stage 3 has been performed without an SHH inhibitor such as cyclopamine in Stage 3. PDX1-positive foregut cells produced from Stage 3 co-express PDX1 and HNF6 as well as SOX9 and PROX, and do not appreciably co-express markers indicative of definitive endoderm or foregut endoderm (PDX1-negative foregut endoderm) cells or PDX1-positive foregut endoderm cells as described above in Stages 1 and 2.

The above stage 3 method is one of four stages for the production of PEC populations. For the production of endocrine progenitor/precursor and endocrine cells as described in detail below, in addition to Noggin, KAAD-cyclopamine and Retinoid; Activin, Wnt and Heregulin, thyroid hormone, TGFb-receptor inhibitors, Protein kinase C activators, Vitamin C, and ROCK inhibitors, alone and/or combined, are used to suppress the early expression NGN3 and increasing CHGA-negative type of cells.

Stage 4 (about days 8-14) PEC culture production takes the media from Stage 3 and exchanges it for media containing DMEM in 1% vol/vol B27 supplement, plus 50 ng/mL KGF and 50 ng/mL of EGF and sometimes also 50 ng/mL Noggin and a ROCK inhibitor and further includes Activin alone or combined with Heregulin. Alternatively, Stage 3 cells can be further differentiated using KGF, RA, SANT, PKC activator and/or Vitamin C or any combination thereof. These methods give rise to pancreatic progenitor cells co-expressing at least PDX1 and NKX6.1 as well as PTF1A. These cells do not appreciably express markers indicative of definitive endoderm or foregut endoderm (PDX1-negative foregut endoderm) cells as described above in Stages 1, 2 and 3.

Stage 5 production takes Stage 4 PEC cell populations above and further differentiates them to produce endocrine progenitor/precursor or progenitor type cells and/or singly and poly-hormonal pancreatic endocrine type cells in a medium containing DMEM with 1% vol/vol B27 supplement, Noggin, KGF, EGF, RO (a gamma secretase inhibitor), nicotinamide and/or ALK5 inhibitor, or any combination thereof, e.g. Noggin and ALK5 inhibitor, for about 1 to 6 days (preferably about 2 days, i.e. days 13-15). Alternatively, Stage 4 cells can be further differentiated using retinoic acid (e.g. RA or an analog thereof), thyroid hormone (e.g. T3, T4 or an analogue thereof), TGFb receptor inhibitor (ALK5 inhibitor), BMP inhibitor (e.g. Noggin, Chordin, LDN), or gamma secretase inhibitor (e.g., XXI, XX, DAPT, XVI, L685458), and/or betacellulin, or any combination thereof. Endocrine progenitor/precursors produced from Stage 5 co-express at least PDX1/NKX6.1 and also express CHGA, NGN3 and Nkx2.2, and do not appreciably express markers indicative of definitive endoderm or foregut endoderm (PDX1-negative foregut endoderm) as described above in Stages 1, 2, 3 and 4 for PEC production.

Stage 6 and 7 can be further differentiated from Stage 5 cell populations by adding any of a combination of agents or factors including but not limited to PDGF+SSH inhibitor (e.g. SANT, cyclopamine, HIP1), BMP inhibitor (e.g. Noggin, Chordin, LDN), nicotinamide, insulin-like growth factor (e.g. IGF1, IGF2), TTNBP, ROCK inhibitor (e.g. Y27632), TGFb receptor inhibitor (e.g. ALK5i), thyroid hormone (e.g. T3, T4 and analogues thereof), and/or a gamma secretase inhibitor (XXI, XX, DAPT, XVI, L685458) or any combination thereof to achieve the cell culture populations or appropriate ratios of endocrine cells, endocrine precursors and immature beta cells.

Stage 7 or immature beta cells are considered endocrine cells but may or may not me sufficiently mature to respond to glucose in a physiological manner. Stage 7 immature beta cells may express MAFB, whereas MAFA and MAFB expressing cells are fully mature cells capable of responding to glucose in a physiological manner.

Stages 1 through 7 cell populations are derived from human pluripotent stem cells (e.g. human embryonic stem cells, induced pluripotent stem cells, genetically modified stem cells e.g. using any of the gene editing tools and applications now available or later developed) and may not have their exact naturally occurring corresponding cell types since they were derived from immortal human pluripotent stem cells generated in vitro (i.e. in an artificial tissue culture) and not the inner cell mass in vivo (i.e. in vivo human development does not have an human ES cell equivalent).

Pancreatic cell therapy replacements as intended herein can be encapsulated in the described herein devices consisting of herein described membranes using any of Stages 4, 5, 6 or 7 cell populations and are loaded and wholly contained in a macro-encapsulation device and transplanted in a patient, and the pancreatic endoderm lineage cells mature into pancreatic hormone secreting cells, or pancreatic islets, e.g., insulin secreting beta cells, in vivo (also referred to as “in vivo function”) and are capable of responding to blood glucose normally.

Encapsulation of the pancreatic endoderm lineage cells and production of insulin in vivo is described in detail in U.S. application Ser. No. 12/618,659 (the '659 Application), entitled ENCAPSULATION OF PANCREATIC LINEAGE CELLS DERIVED FROM HUMAN PLURIPOTENT STEM CELLS, filed Nov. 13, 2009. The '659 Application claims the benefit of priority to Provisional Patent Application No. 61/114,857, entitled ENCAPSULATION OF PANCREATIC PROGENITORS DERIVED FROM HES CELLS, filed Nov. 14, 2008; and U.S. Provisional Patent Application No. 61/121,084, entitled ENCAPSULATION OF PANCREATIC ENDODERM CELLS, filed Dec. 9, 2008; and now U.S. Pat. Nos. 8,278,106 and 8,424,928. The methods, compositions and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. Accordingly, it will be apparent to one skilled in the art that varying substitutions and modifications may be made to the invention disclosed herein without departing from the scope and spirit of the invention.

Additionally, embodiments described herein are not limited to any one type of pluripotent stem cell or human pluripotent stem cell and include but are not limited to human embryonic stem (hES) cells and human induced pluripotent stem (iPS) cells or other pluripotent stem cells later developed. It is also well known in the art, that as of the filing of this application, methods for making human pluripotent stems may be performed without destruction of a human embryo and that such methods are anticipated for production of any human pluripotent stem cell.

Methods for producing pancreatic cell lineages from human pluripotent cells were conducted substantially as described in at least the listed publications assigned to ViaCyte, Inc. including but not limited to: PCT/US2007/62755 (WO2007101130), PCT/US2008/80516 (WO2009052505), PCT/US2008/82356 (WO2010053472), PCT/US2005/28829 (WO2006020919), PCT/US2014/34425 (WO2015160348), PCT/US2014/60306 (WO2016080943), PCT/US2016/61442 (WO2018089011), PCT/US2014/15156 (WO2014124172), PCT/US2014/22109 (WO2014138691), PCT/US2014/22065 (WO2014138671), PCT/US2005/14239 (WO2005116073), PCT/US2004/43696 (WO2005063971), PCT/US2005/24161 (WO2006017134), PCT/US2006/42413 (WO2007051038), PCT/US2007/15536 (WO2008013664), PCT/US2007/05541 (WO2007103282), PCT/US2008/61053 (WO2009131568), PCT/US2008/65686 (WO2009154606), PCT/US2014/15156 (WO2014124172), PCT/US2018/41648 (WO2019014351), PCT/US2014/26529 (WO2014160413), PCT/US2009/64459 (WO2010057039); and d'Amour et al. 2005 Nature Biotechnology 23:1534-41; D'Amour et al. 2006 Nature Biotechnology 24(11):1392-401; McLean et al., 2007 Stem Cells 25:29-38, Kroon et al. 2008 Nature Biotechnology 26(4): 443-452, Kelly et al. 2011 Nature Biotechnology 29(8): 750-756, Schulz et al., 2012 PLos One 7(5):e37004; and/or Agulnick et al. 2015 Stem Cells Transl. Med. 4(10):1214-22.

Methods for producing pancreatic cell lineages from human pluripotent cells were conducted substantially as described in at least the listed below publications assigned to Janssen including but not limited to: PCT/US2008/68782 (WO200906399), PCT/US2008/71775 (WO200948675), PCT/US2008/71782 (WO200918453), PCT/US2008/84705 (WO200970592), PCT/US2009/41348 (WO2009132063), PCT/US2009/41356 (WO2009132068), PCT/US2009/49183 (WO2010002846), PCT/US2009/61635 (WO2010051213), PCT/US2009/61774 (WO2010051223), PCT/US2010/42390 (WO2011011300), PCT/US2010/42504 (WO2011011349), PCT/US2010/42393 (WO2011011302), PCT/US2010/60756 (WO2011079017), PCT/US2011/26443 (WO2011109279), PCT/US2011/36043 (WO2011143299), PCT/US2011/48127 (WO2012030538), PCT/US2011/48129 (WO2012030539), PCT/US2011/48131 (WO2012030540), PCT/US2011/47410 (WO2012021698), PCT/US2012/68439 (WO2013095953), PCT/US2013/29360 (WO2013134378), PCT/US2013/39940 (WO2013169769), PCT/US2013/44472 (WO2013184888), PCT/US2013/78191 (WO2014106141), PCT/US2014/38993 (WO2015065524), PCT/US2013/75939 (WO2014105543), PCT/US2013/75959 (WO2014105546), PCT/US2015/29636 (WO2015175307), PCT/US2015/64713 (WO2016100035), PCT/US2014/41988 (WO2015002724), PCT/US2017/25847 (WO2017180361), PCT/US2017/37373 (WO2017222879), PCT/US2017/37373 (WO2017222879); P CT/U S2009/049049 (WO2010/002785), PCT/US2010/060770 (WO2011/079018), PCT/US2014/042796, (WO2015/065537), PCT/US2008/070418 (WO2009/012428); Bruin et al. 2013 Diabetologia. 56(9): 1987-98, Fryer et al. 2013 Curr. Opin. Endocrinol. Diabetes Obes. 20(2): 112-7, Chetty et al. 2013 Nature Methods. 10(6):553-6, Rezania et al. 2014 Nature Biotechnologyy 32(11):1121-33, Bruin et al. 2014 Stem Cell Res. 12(1): 194-208, Hrvatin 2014 Proc. Natl. Acad. Sci. USA. 111(8): 3038-43, Bruin et al. 2015 Stem Cell Reports. 5, 1081-1096, Bruin et al. 2015 Science Transl. Med., 2015, 7, 316ps23, and/or Bruin et al. 2015 Stem Cell Reports. 14; 4(4):605-20.

In one embodiment, human pluripotent cells were differentiated to PDX1-positive pancreatic endodermcells including pancreatic progenitors and endocrine precursors according to one of the preferred following conditions A and/or B.

TABLE 1
Media Conditions for PDX1-positive Pancreatic
Endoderm Cell Production

| Stage | A | B |
|---|---|---|
| 1 | r0.2FBS-ITS1:5000 A100 W50 | |
| | r0.2FBS-ITS1:5000 A100 | |
| 2 | r0.2FBS-ITS1:1000 K25 IV | |
| | r0.2FBS-ITS1:1000 K25 | |
| | r0.2FBS-ITS1:1000 K25 | |
| 3 | db-TT3 N50 | |
| | db-TT3 N50 | |
| | db-TT3 N50 | |
| 4 | db-N50 K50 E50 | |
| | db-N50 K50 E50 | |
| | db-N50 K50 E50 | |
| | db-N50 K50 E50 --> Cryopreserved | |
| Thaw | db-N50 K50 E50 | db-N100 A5i (1 uM) |
| (S5- | db-N50 K50 E50 | db-N100 A5i (1 uM) |
| S6) | db-N50 K50 E50 | db-N100 A5i (1 uM) |
| | | db-N100 A5i (10 uM) |
| | | db-A5i (10 uM) |
| | | db-A5i (10 uM) |

Table 1 Legend: r0.2FBS: RPMI 1640 (Mediatech); 0.2% FBS (HyClone), 1× GlutaMAX-1 (Life Technologies), 1% v/v penicillin/streptomycin; db: DMEM Hi Glucose (HyClone) supplemented with 0.5x B-27 Supplement (Life Technologies); A100, A50, A5: 100 ng/mL recombinant human Activin A (R&D Systems); A5i: 1 uM, 5 uM, 10 uM ALK5 inhibitor; TT3: 3 nM TTNPB (Sigma-Aldrich); E50: 50 ng/mL recombinant human EGF (R&D Systems); ITS: Insulin-Transferrin-Selenium (Life Technologies) diluted 1:5000 or 1:1000; IV: 2.5 mM TGF-b RI Kinase inhibitor IV (EMD Bioscience); K50, K25: 50 ng/mL, 25 ng/mL recombinant human KGF (R&D Systems, or Peprotech); N50, N100: 50 ng/mL or 100 ng/mL recombinant human Noggin (R&D Systems); W50: 50 ng/mL recombinant mouse Wnt3A (R&D Systems).

One of ordinary skill in the art will appreciate that there may exist other methods for production of PDX1-positive pancreatic endoderm cells or PDX1-positive pancreatic endoderm lineage cells including pancreatic progenitors or even endocrine and endocrine precursor cells; and at least those PDX1-positive pancreatic endoderm cells described in Kroon et al. 2008, Rezania et al. 2014 supra and Pagliuca et al. 2014 Ce11159(2):428-439, supra.

One of ordinary skill in the art will also appreciate that the embodiments described herein for production of PDX1-positive pancreatic endoderm cells consists of a mixed population or a mixture of subpopulations. And because unlike mammalian in vivo development which occurs along the anterior-posterior axis, and cells and tissues are named such accordingly, cell cultures in any culture vessels lack such directional patterning and thus have been characterized in particular due to their marker expression. Hence, mixed subpopulations of cells at any stage of differentiation does not occur in vivo. The PDX1-positive pancreatic endoderm cell cultures therefore include, but are not limited to: i) endocrine precursors (as indicated e.g. by the early endocrine marker, Chromogranin A or CHGA); ii) singly hormonal polyhormonal cells expressing any of the typical pancreatic hormones such as insulin (INS), somatostatin (SST), pancreatic polypeptide (PP), glucagon (GCG), or even gastrin, incretin, secretin, or cholecystokinin; iii) pre-pancreatic cells, e.g. cells that express PDX-1 but not NKX6.1 or CHGA; iv) endocrine cells that co-express PDX-1/NKX6.1 and CHGA (PDX-1/NKX6.1/CHGA), or non-endocrine e.g., PDX-1/NKX6.1 but not CHGA (PDX-1+/NKX6.1+/CHA−); and v) still there are cells that do not express PDX-1, NKX6.1 or CHGA (e.g. triple negative cells).

This PDX1-positive pancreatic endoderm cells population with its mixed subpopulations of cells mostly express at least PDX-1, in particular a subpopulation that expresses PDX-1/NKX6.1. The PDX1/NKX6.1 subpopulation has also been referred to as “pancreatic progenitors”, “Pancreatic Epithelium” or “PEC” or versions of PEC, e.g. PEC-01. Although Table 1 describes a stage 4 population of cells, these various subpopulations are not limited to just stage 4. Certain of these subpopulations can be for example found in as early as stage 3 and in later stages including stages 5, 6 and 7 (immature beta cells). The ratio of each subpopulation will vary depending on the cell culture media conditions employed. For example, in Agulnick et al. 2015, supra, 73-80% of PDX-1/NKX6.1 cells were used to further differentiate to islet-like cells (ICs) that contained 74-89% endocrine cells generally, and 40-50% of those expressed insulin (INS). Hence, different cell culture conditions are capable of generating different ratios of subpopulations of cells and such may effect in vivo function and therefore blood serum c-peptide levels. And whether modifying methods for making PDX1-positive pancreatic endoderm lineage cell culture populations effects in vivo function can only be determined using in vivo studies as described in more detail below. Further, it cannot be assumed and should not be assumed that just because a certain cell type has been made and has well characterized, that such a method produces the same cell intermediates, unless this is also well characterized.

In one aspect, a method for producing mature beta cells in vivo is provided. The method consisting of making human definitive endoderm lineage cells derived from human pluripotent stem cells in vitro with at least a TGFβ superfamily member and/or at least a TGFβ superfamily member and a Wnt family member, preferably a TGFβ superfamily member and a Wnt family member, preferably Activin A, B or GDF-8, GDF-11 or GDF-15 and Wnt3a, preferably Actvin A and Wnt3a, preferably GDF-8 and Wnt3a. The method for making PDX1-positive pancreatic endoderm cells from definitive endoderm cells with at least KGF, a BMP inhibitor and a retinoic acid (RA) or RA analog, and preferably with KGF, Noggin and RA. The method may further differentiate the PDX1-positive pancreatic endoderm cells into immature beta cells or MAFA expressing cells with a thyroid hormone and/or a TGFb-RI inhibitor, a BMP inhibitor, KGF, EGF, a thyroid hormone, and/or a Protein Kinase C activator; preferably with noggin, KGF and EGF, preferably additionally with T3 or T4 and ALK5 inhibitor or T3 or T4 alone or ALK5 inhibitor alone, or T3 or T4, ALK5 inhibitor and a PKC activator such as ILV, TPB and PdBu. Or preferably with noggin and ALK5i and implanting and maturing the PDX1-positive pancreatic endoderm cells or the MAFA immature beta cell populations into a mammalian host in vivo to produce a population of cells including insulin secreting cells capable of responding to blood glucose.

In one aspect, a unipotent human immature beta cell or PDX1-positive pancreatic endoderm cell that expresses INS and NKX6.1 and does not substantially express NGN3 is provided. In one embodiment, the unipotent human immature beta cell is capable of maturing to a mature beta cell. In one embodiment, the unipotent human immature beta cell further expresses MAFB in vitro and in vivo. In one embodiment, the immature beta cells express INS, NKX6.1 and MAFA and do not substantially express NGN3.

In one aspect, pancreatic endoderm lineage cells expressing at least CHGA (or CHGA+) refer to endocrine cells; and pancreatic endoderm cells that do not express CHGA (or CHGA−) refer to non-endocrine cells. In another aspect, these endocrine and non-endocrine sub-populations may be multipotent progenitor/precursor sub-populations such as non-endocrine multipotent pancreatic progenitor sub-populations or endocrine multipotent pancreatic progenitor sub-populations; or they may be unipotent sub-populations such as immature endocrine cells, preferably immature beta cells, immature glucagon cells and the like.

In one aspect, more than 10% preferably more than 20%, 30%, 40% and more preferably more than 50%, 60%, 70%, 80%, 90%, 95%, 98% or 100% of the cells in the pancreatic endoderm or PDX1-positive pancreatic endoderm cell population (stage 4) are the non-endocrine (CHGA−) multipotent progenitor sub-population that give rise to mature insulin secreting cells and respond to glucose in vivo when implanted into a mammalian host.

One embodiment provides a composition and method for differentiating pluripotent stem cells in vitro to substantially pancreatic endoderm cultures and further differentiating the pancreatic endoderm culture to endocrine or endocrine precursor cells in vitro. In one aspect, the endocrine precursor or endocrine cells express CHGA. In one aspect, the endocrine cells can produce insulin in vitro. In one aspect, the in vitro endocrine insulin secreting cells may produce insulin in response to glucose stimulation. In one aspect, more than 10% preferably more than 20%, 30%, 40% and more preferably more than 50%, 60%, 70%, 80%, 90%, 95%, 98% or 100% of the cells in the cells population are endocrine cells.

Embodiments described herein provide for compositions and methods of differentiating pluripotent human stem cells in vitro to endocrine cells. In one aspect, the endocrine cells express CHGA. In one aspect, the endocrine cells can produce insulin in vitro. In one aspect, the endocrine cells are immature endocrine cells such as immature beta cells. In one aspect, the in vitro insulin producing cells may produce insulin in response to glucose stimulation.

One embodiment provides a method for producing insulin in vivo in a mammal, the method comprising: (a) loading a pancreatic endoderm cell or endocrine cell or endocrine precursor cell population into an implantable semi-permeable device; (b) implanting the device with the cell population into a mammalian host; and (c) maturing the cell population in the device in vivo wherein at least some of the endocrine cells are insulin secreting cells that produce insulin in response to glucose stimulation in vivo, thereby producing insulin in vivo to the mammal. In one aspect the endocrine cell is derived from a cell composition comprising PEC with a higher non-endocrine multipotent pancreatic progenitor sub-population (CHGA−). In another aspect, the endocrine cell is derived from a cell composition comprising PEC with a reduced endocrine sub-population (CHGA+). In another aspect, the endocrine cell is an immature endocrine cell, preferably an immature beta cell.

In one aspect the endocrine cells made in vitro from pluripotent stem cells express more PDX1 and NKX6.1 as compared to PDX-1 positive pancreatic endoderm populations, or the non-endocrine (CHGA−) subpopulations which are PDX1/NKX6.1 positive. In one aspect, the endocrine cells made in vitro from pluripotent stem cells express PDX1 and NKX6.1 relatively more than the PEC non-endocrine multipotent pancreatic progenitor sub-population (CHGA−). In one aspect, a Bone Morphogenic Protein (BMP) and a retinoic acid (RA) analog alone or in combination are added to the cell culture to obtain endocrine cells with increased expression of PDX1 and NKX6.1 as compared to the PEC non-endocrine multipotent progenitor sub-population (CHGA−). In one aspect BMP is selected from the group comprising BMP2, BMP5, BMP6, BMP7, BMP8 and BMP4 and more preferably BMP4. In one aspect the retinoic acid analog is selected from the group comprising all-trans retinoic acid and TTNPB (4-[(E)-2-(5,6,7,8-Tetrahydro-5,5,8,8-tetramethyl-2-naphthalenyl)-I-propenyl]benzoic acid Arotinoid acid), or 0.1-10 μM AM-580 (4-[(5,6,7,8-Tetrahydro-5,5,8,8-tetramethyl-2-naphthalenyl)carboxamido]benzoic acid) and more preferably TTNPB.

One embodiment provides a method for differentiating pluripotent stem cells in vitro to endocrine and immature endocrine cells, preferably immature beta cells, comprising dissociating and re-associating the aggregates. In one aspect the dissociation and re-association occurs at stage 1, stage 2, stage 3, stage 4, stage 5, stage 6 or stage 7 or combinations thereof. In one aspect the definitive endoderm, PDX1-negative foregut endoderm, PDX1-positive foregut endoderm, PEC, and/or endocrine and endocrine progenitor/precursor cells are dissociated and re-associated. In one aspect, the stage 7 dissociated and re-aggregated cell aggregates consist of fewer non-endocrine (CHGA−) sub-populations as compared to endocrine (CHGA+) sub-populations. In one aspect, more than 10% preferably more than 20%, 30%, 40% and more preferably more than 50%, 60%, 70%, 80%, 90%, 95%, 98% or 100% of the cells in the cell population are endocrine (CHGA+) cells.

One embodiment provides a method for differentiating pluripotent stem cells in vitro to endocrine cells by removing the endocrine cells made during stage 4 PEC production thereby enriching for non-endocrine multipotent pancreatic progenitor (CHGA−) sub-population which is PDX1+ and NKX6.1+.

In one embodiment, PEC cultures enriched for the non-endocrine multipotent progenitor sub-population (CHGA−) are made by not adding a Noggin family member at stage 3 and/or stage 4. In one embodiment, PEC cultures which are relatively replete of cells committed to the endocrine lineage (CHGA+) are made by not adding a Noggin family member at stage 3 and/or stage 4. In one aspect the Noggin family member is a compound selected from the group comprising Noggin, Chordin, Follistatin, Folistatin-like proteins, Cerberus, Coco, Dan, Gremlin, Sclerostin, PRDC (protein related to Dan and Cerberus).

One embodiment provides a method for maintaining endocrine cells in culture by culturing them in a media comprising exogenous high levels of glucose, wherein the exogenous glucose added is about 1 mM to 25 mM, about 1 mM to 20 mM, about 5 mM to 15 mM, about 5 mM to 10 mM, about 5 mM to 8 mM. In one aspect, the media is a DMEM, CMRL or RPMI based media.

One embodiment provides a method for differentiating pluripotent stem cells in vitro to endocrine cells with and without dissociating and re-associating the cell aggregates. In one aspect the non-dissociated or the dissociated and re-associated cell aggregates are cryopreserved or frozen at stage 6 and/or stage 7 without affecting the in vivo function of the endocrine cells. In one aspect, the cryopreserved endocrine cell cultures are thawed, cultured and, when transplanted, function in vivo.

Another embodiment provides a culture system for differentiating pluripotent stem cells to endocrine cells, the culture system comprising of at least an agent capable of suppressing or inhibiting endocrine gene expression during early stages of differentiation and an agent capable of inducing endocrine gene expression during later stages of differentiation. In one aspect, an agent capable of suppressing or inhibiting endocrine gene expression is added to the culture system consisting of pancreatic PDX1 negative foregut cells. In one aspect, an agent capable of inducing endocrine gene expression is added to the culture system consisting of PDX1-positive pancreatic endoderm progenitors or PEC. In one aspect, an agent capable of suppressing or inhibiting endocrine gene expression is an agent that activates a TGFbeta receptor family, preferably it is Activin, preferably, it is high levels of Activin, followed by low levels of Activin. In one aspect, an agent capable of inducing endocrine gene expression is a gamma secretase inhibitor selected from a group consisting of N—[N-(3,5-Diflurophenacetyl-L-alanyl)]-S-phenylglycine t-Butyl Ester (DAPT), R044929097, DAPT (N—[N-(3,5-Difluorophenacetyl-L-alanyl)]-S-phenylglycine t-Butyl Ester), 1-(S)-endo-N-(1,3,3)-Trimethylbicyclo[2.2.1]hept-2-yl)-4-fluorophenyl Sulfonamide, WPE-III31C, S-3-[N′-(3,5-difluorophenyl-alpha-hydroxyacetyl)-L-alanilyl]amino-2,3-dih-ydro-1-methyl-5-phenyl-1H-1,4-benzodiazepin-2-one, (N)—[(S)-2-hydroxy-3-methyl-butyryl]-1-(L-alaninyl)-(S)-1-amino-3-methyl-4,5,6,7-tetrahydro-2H-3-benzazepin-2-one, BMS-708163 (Avagacestat), BMS-708163, Semagacestat (LY450139), Semagacestat (LY450139), MK-0752, MK-0752, Y0-01027, YO-01027 (Dibenzazepine, DBZ), LY-411575, LY-411575, or LY2811376. In one aspect, high levels of Activin is meant levels greater than 40 ng/mL, 50 ng/mL, and 75 ng/mL. In one aspect, high levels of Activin are used during stage 3 or prior to production of pancreatic foregut endoderm cells. In one aspect, low levels of Activin means less than 30 ng/mL, 20 ng/mL, 10 ng/mL and 5 ng/mL. In one aspect, low levels of Activin are used during stage 4 or for production of PEC. In one aspect, the endocrine gene that is inhibited or induced is NGN3. In another aspect, Activin A and Wnt3A are used alone or in combination to inhibit endocrine expression, preferably to inhibit NGN3 expression prior to production of pancreatic foregut endoderm cells, or preferably during stage 3. In one aspect, a gamma secretase inhibitor, preferably R044929097 or DAPT, is used in the culture system to induce expression of endocrine gene expression after production of PEC, or preferably during stages 5, 6 and/or 7.

An in vitro cell culture comprising endocrine cells wherein at least 5% of the human cells express an endocrine marker selected from the group consisting of, insulin (INS), NK6 homeobox 1(NKX6.1), pancreatic and duodenal homeobox 1 (PDX1), transcription factor related locus 2 (NKX2.2), paired box 4 (PAX4), neurogenic differentiation 1 (NEUROD), forkhead box A1 (FOXA1), forkhead box A2 (FOXA2), snail family zinc finger 2 (SNAIL2), and musculoaponeurotic fibrosarcoma oncogene family A and B (MAFA and MAFB), and does not substantially express a marker selected from the group consisting of neurogenin 3 (NGN3), islet 1 (ISL1), hepatocyte nuclear factor 6 (HNF6), GATA binding protein 4 (GATA4), GATA binding protein 6 (GATA6), pancreas specific transcription factor 1a (PTF1A) and SRY (sex determining region Y)-9 (SOX9), wherein the endocrine cells are unipotent and can mature to pancreatic beta cells.

### EXAMPLES

### Example 1

A two layer bonded composite was created by thermally bonding two discrete layers together

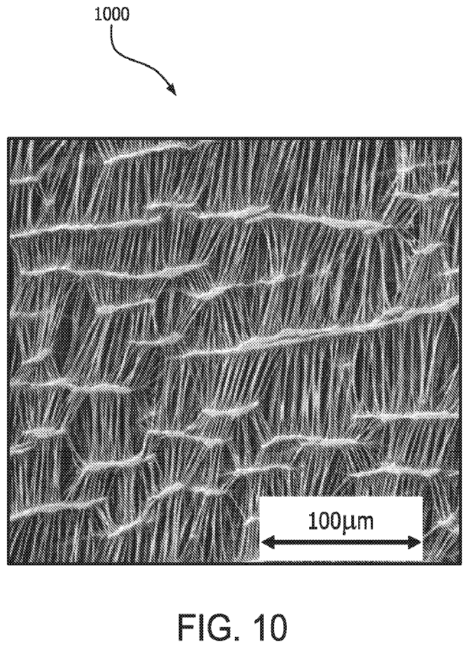

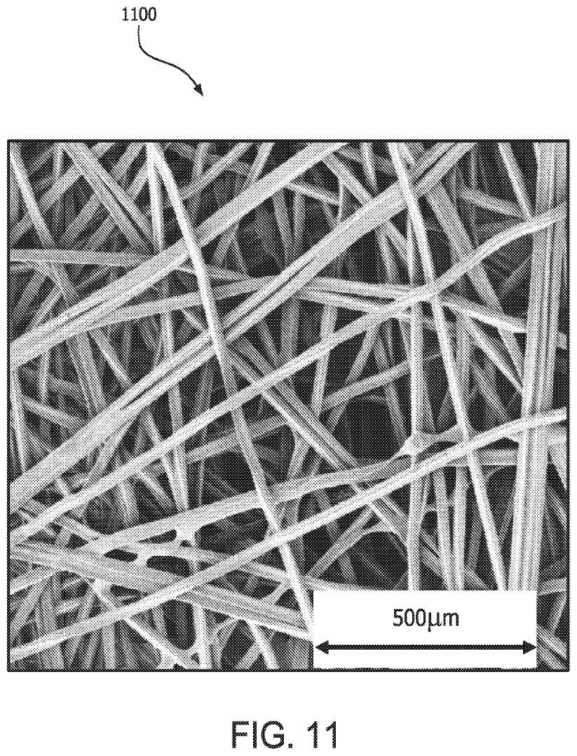

The first layer of the two layer biocompatible membrane composite was an expanded polytetrafluoroethylene membrane (ePTFE) (Mitigation Layer) prepared according to the teachings of U.S. Pat. No. 5,814,405 to Branca, et al. The scanning electron micrograph (SEM) image shown in FIG. 10 is a representative image of the surface of the ePTFE membrane of the first layer (i.e., Mitigation Layer). The properties of this ePTFE layer are shown in Table 1. The second layer was a commercially acquired spunbond polyester non-woven material (Vascularization Layer). A representative surface microstructure of the third layer is shown in the SEM image shown in FIG. 11. The properties of this layer are shown in Table 1.

TABLE 1

| | Layer Function | |
|---|---|---|
| | FBGC | |
| | Mitigation | Vascularization |
| | ePTFE | PET Non- |
| Description | Open Layer | woven |
| Pore Size (microns) | 8.1 | 102 |
| Thickness (microns) | 44.6 | 77.4 |
| Mass (g/m²) | 6.2 | 12.4 |
| Porosity (%) | 93.7 | 92.7 |
| Solid Feature Spacing (microns) | 25.7 | 89.4 |
| Solid Feature Minor Axis | 7.8 | 32.2 |
| (microns) | | |
| Solid Feature Major Axis | 71.2 | — |
| (microns) | | |
| Solid Feature Depth (microns) | 15.3 | 29.9 |

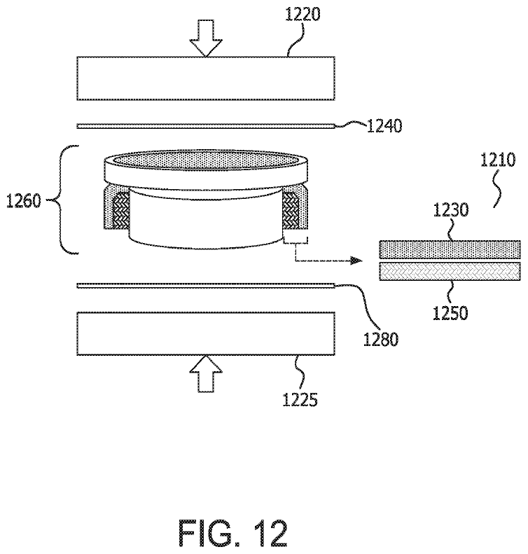

The mitigation layer and the vascularization layer were bonded together by laying them up adjacent to each other and restraining them within an aluminum tensioning ring with an aluminum backing block. The vascularization layer (non-woven layer) was oriented such that it was touching the aluminum backing block. The ePTFE membrane was facing outwards in the tensioning hoop. The materials in the tensioning ring with a backing block were then sandwiched between two steel plates and placed in a carver press. FIG. 12 illustrates an exploded view of the configuration of materials used. In particular, the materials included a carver press top platen 1220, a top steel plate 1240, a tension ring with backing block 1260, a bottom steel plate 1280, and a carver press bottom platen 1225. The two layer biocompatible membrane composite 1210 included the first layer (Mitigation Layer) 1230 and second layer (Vascularization Layer) 1250.

A carver press was set to a temperature of 235° C. and minimal pressure was applied so that the carver press platens were in contact with the steel plates but no pressure registered on the pressure gauge. After a dwell time of 45 seconds, contact from the carver press platens was removed. When the mitigation and vascularization layers were removed from the tensioning ring, they were bonded together as a biocompatible membrane composite.

### Example 2

Three biocompatible membrane composites, each having two distinct layers each were constructed in a similar manner. The three constructs shared similar first layers (Mitigation Layers) but had different second layers (Vascularization Layers). The three different biocompatible membrane composites will henceforth be referred to as Construct A, Construct B, and Construct C.

The first expanded polytetrafluoroethylene (ePTFE) membrane was prepared according to the teachings of U.S. Pat. No. 5,814,405 to Branca, et al. The ePTFE tape precursor of the first ePTFE layer was processed per the teachings of U.S. Pat. No. 5,814,405 to Branca, et al. through the below-the-melt machine direction (MD) expansion step. During the below-the-melt MD expansion step of the first ePTFE tape precursor, an FEP film was applied per the teachings of WO 94/13469 to Bacino. The ePTFE tape precursor of the second ePTFE layer was processed per the teachings of U.S. Pat. No. 5,814,405 to Branca, et al. through an amorphous locking step and above-the-melt MD expansion. The properties of the tape precursor and amount of MD expansion performed on the second layer varied between the three constructs. During the first below-the-melt MD expansion step of the second ePTFE tape precursor, an FEP film was applied per the teachings of WO 94/13469 to Bacino. The expanded ePTFE tape precursor of the second ePTFE membrane was laminated to the expanded ePTFE tape precursor of the first ePTFE membrane such that the FEP side of the second ePTFE tape was in contact with the PTFE side of the ePTFE tape precursor of the first ePTFE membrane.

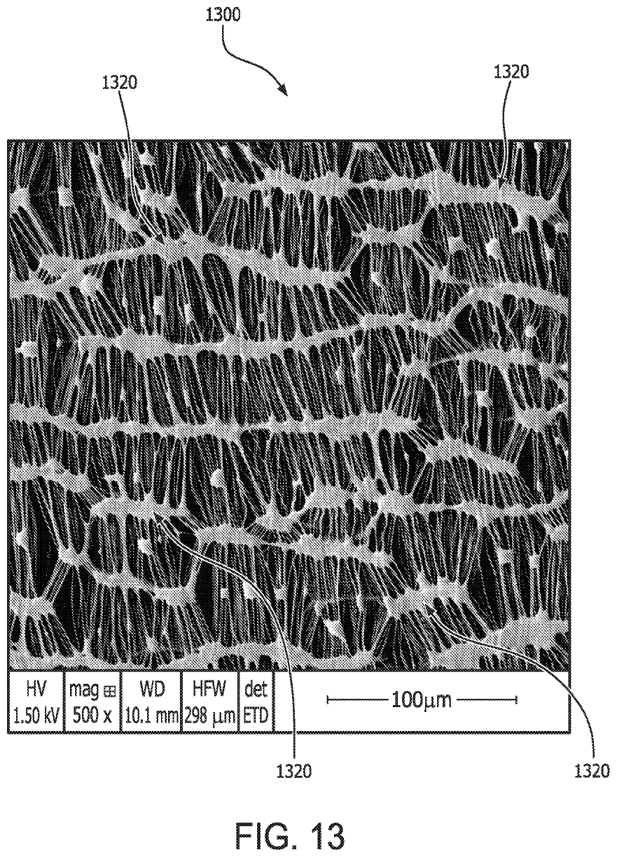

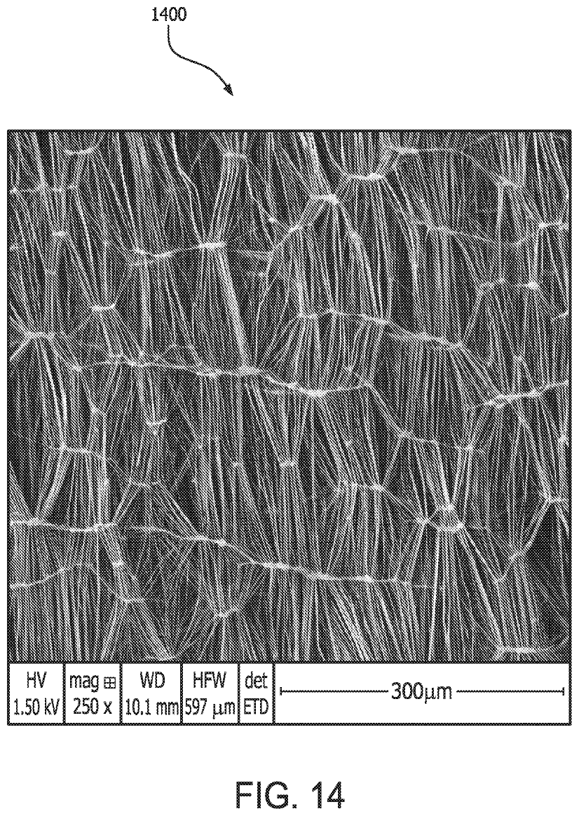

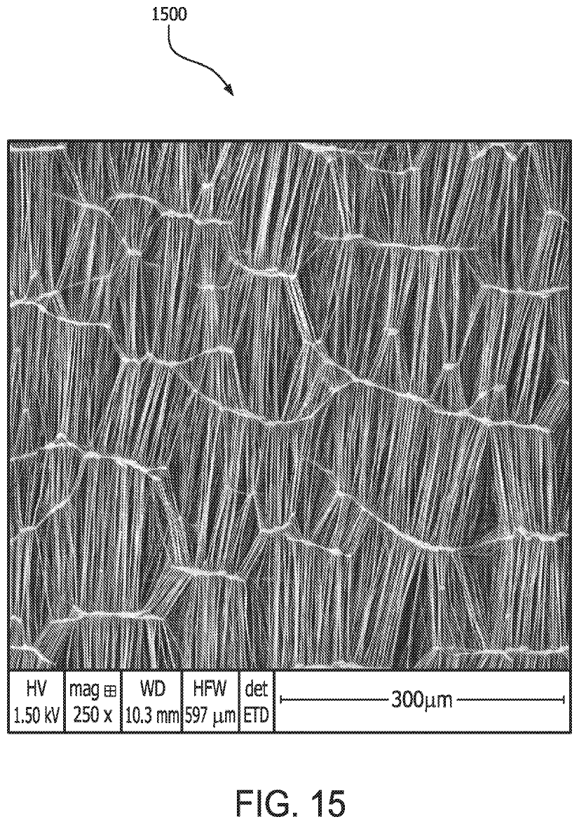

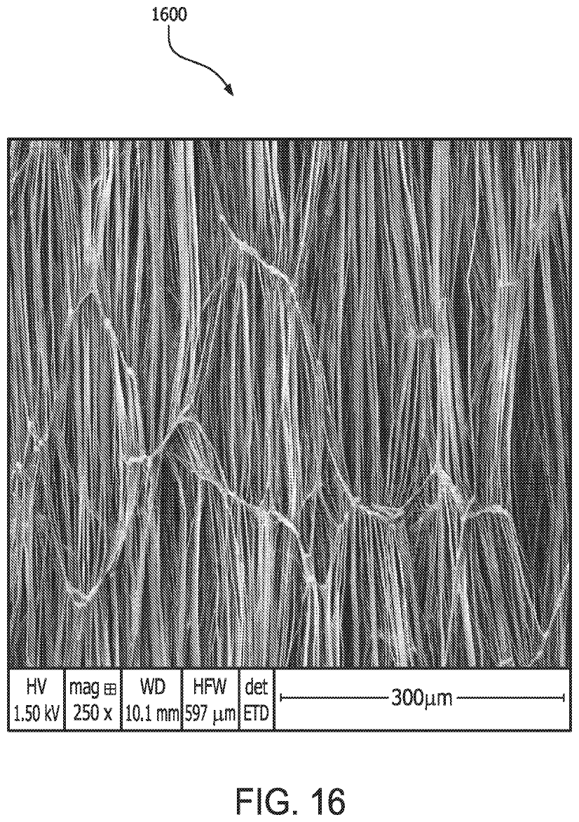

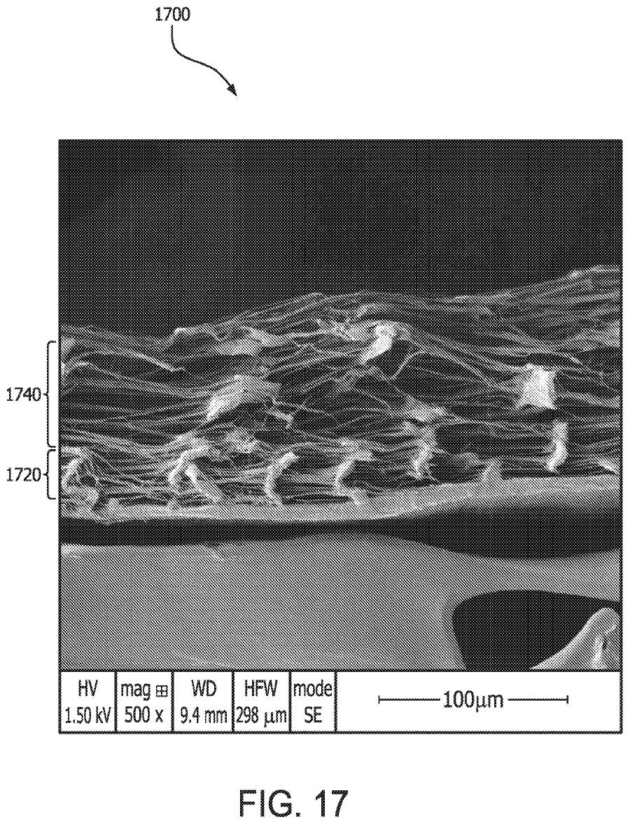

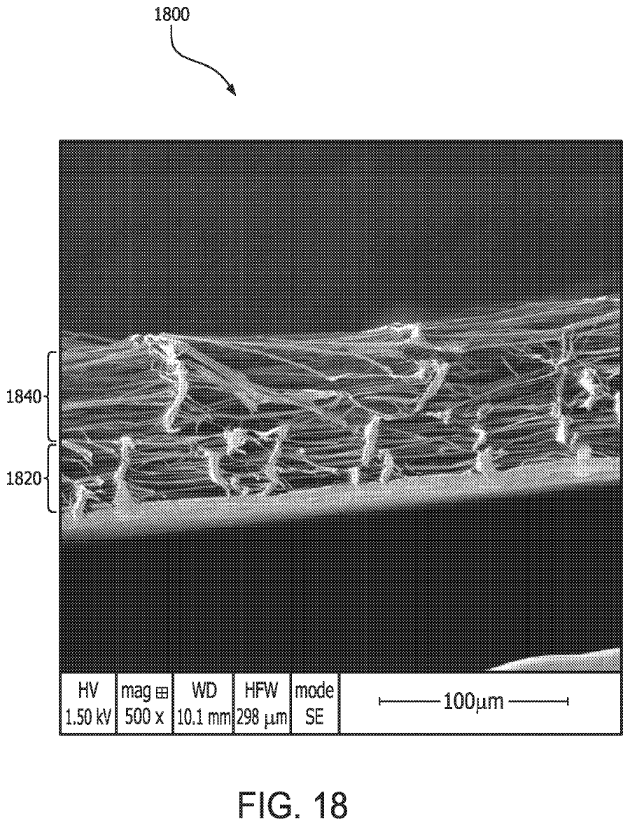

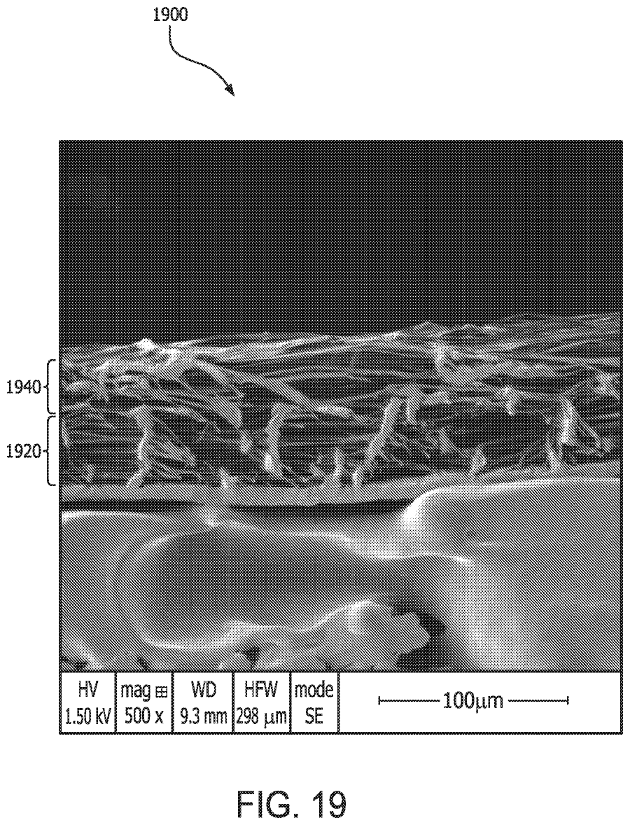

The two layer biocompatible membrane composite was then co-expanded in the machine direction and transverse direction above the melting point of PTFE. A representative surface microstructure of the first ePTFE layer having thereon FEP 1320 is shown in the scanning electron micrograph (SEM) image of FIG. 13. The SEM images shown in FIG. 14, FIG. 15, and FIG. 16 are representative images of the node and fibril structure of the second ePTFE membranes 1400, 1500, and 1600 (Vascularization Layers), respectively. The SEM images shown in FIG. 17, FIG. 18, and FIG. 19 are representative images of the cross-section structures of the two layer biocompatible membrane composites including the first ePTFE membranes 1720, 1820, and 1920 (Mitigation Layers), respectively, and the second ePTFE membranes 1740, 1840, and 1940 (Vascularization Layers), respectively.

### Characterization of the Biocompatible Membrane Composite

Each individual layer of the biocompatible membrane composites was evaluated and characterized for the relevant parameters for the function of each layer. The methods used for the characterization of the relevant parameters were performed in accordance with the test methods described in the Test Methods section set forth above. The results are summarized in Table 2.

TABLE 2

| | Construct | | | |
|---|---|---|---|---|
| | All | Construct A | Construct B | Construct C |
| | Layer Function | | | |
| | FBGC | Vascular- | Vascular- | Vascular- |
| | Mitigation | ization | ization | ization |
| | Layer | A | B | C |
| | ePTFE | ePTFE | ePTFE | ePTFE |
| | Open | Open | Open | Open |
| Description | Layer | Layer | Layer | Layer |
| Pore Size (μm) | 7.36-8.85 | 10.24 | 14.45 | 20.15 |
| Thickness (μm) | 33.46-43.71 | 57.4 | 46.47 | 31.17 |
| Mass (g/m²) | 5.7-6.7 | 7.8 | 7.6 | 6.7 |
| Porosity (%) | 90.9-93.8 | 93.8 | 92.6 | 90.2 |
| Solid Feature | 19.6-25.9 | 61.4 | 61.7 | 88.5 |
| Spacing (μm) | | | | |
| Solid Feature | | | | |
| Minor Axis | 7.7-10.1 | 3.6 | 6.0 | 8.8 |
| (μm) | | | | |
| Solid Feature | 27.8-68.1 | 21.8 | 31.3 | 30.8 |
| Major Axis (μm) | | | | |
| Solid Feature | 14.0-20.8 | 13.8 | 19.2 | 11.9 |
| Depth (μm) | | | | |

*Note
that the values listed under each construct were measured after the two layer composite was bonded together, not the vascularization layer alone.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A biocompatible membrane composite comprising:
a first layer having first solid features with a first solid feature spacing, wherein a majority of the first solid feature spacing of the first solid features is less than about 50 microns; and
a second layer having second solid features with a second solid feature spacing, wherein a majority of the second solid feature spacing of the second solid features is greater than about 50 microns.

2. The biocompatible membrane composite of claim 1, wherein the first layer comprises a representative minor axis from about 3 microns to about 20 microns.

3. (canceled)

4. The biocompatible membrane composite of claim 1, wherein the first layer has a first thickness less than about 200 microns.

5. (canceled)

6. The biocompatible membrane composite of claim 1, wherein at least one of the first solid features of the first layer and the second solid features of the second layer are connected by fibrils and the fibrils are deformable.

7. The biocompatible membrane composite of claim 4, wherein the second layer has a second thickness from about 30 microns to about 200 microns.

8. (canceled)

9. The biocompatible membrane composite of claim 1, wherein the biocompatible membrane composite has thereon a surface coating comprising one or more members selected from antimicrobial agents, antibodies, pharmaceuticals and biologically active molecules.

10. The biocompatible membrane composite of claim 1, wherein at least one of the first layer and the second layer is a fluoropolymer membrane.

11. The biocompatible membrane composite of claim 1, wherein the second layer is a spunbound non-woven polyester material.

12. The biocompatible membrane composite of claim 1, comprising a reinforcing component.

13. The biocompatible membrane composite of claim 12, wherein the reinforcing component is a woven or non-woven textile.

14. The biocompatible membrane composite of claim 1,
wherein the first solid features comprise a representative minor axis, a representative major axis and a solid feature depth, and
wherein a majority of the first solid features of the first layer has at least two of the representative minor axis, the representative major axis, and the solid feature depth are greater than about 5 microns.

15. A biocompatible membrane composite comprising:
a first layer having a first thickness less than about 200 microns and first solid features, wherein a majority of a first solid feature spacing of the first solid features is less than about 50 microns; and
a second layer,
wherein a majority of the first solid features has a first representative minor axis from about 3 microns to about 20 microns.

16. (canceled)

17. The biocompatible membrane composite of claim 15, wherein the second layer comprises second solid features and a second solid feature spacing, wherein a majority of the second solid feature spacing of the second solid features is greater than about 50 microns.

18. The biocompatible membrane composite of claim 15, wherein the second layer has a second thickness from about 30 microns to about 200 microns.

19. The biocompatible membrane composite of claim 15, wherein the first solid features include a first representative minor axis, a first representative major axis and a first solid feature depth, and
wherein a majority of the first solid features of the first layer has at least two of the first representative minor axis, the first representative major axis, and the first solid feature depth are greater than about 5 microns.

20. The biocompatible membrane composite of claim 15, wherein the first solid features are connected by fibrils and the fibrils are deformable.

21. The biocompatible membrane composite of claim 15, wherein the second layer comprises second solid features and a majority of the second solid features has a second representative minor axis that is less than about 40 microns.

22. (canceled)

23. (canceled)

24. The biocompatible membrane composite of claim 15, wherein the second layer is a spunbound non-woven polyester material.

25. (canceled)

26. The biocompatible membrane composite of claim 15, wherein the first solid features of the first layer comprise a member selected from thermoplastic polymers, polyurethanes, silicones, rubbers, epoxies and combinations thereof.

27. The biocompatible membrane composite of claim 15, further comprising a reinforcing component.

28. The biocompatible membrane composite of claim 27, wherein the reinforcing component is a woven or non-woven textile.

29. The biocompatible membrane composite of claim 15, wherein the biocompatible membrane composite has thereon a surface coating
comprising one or more members selected from antimicrobial agents, antibodies, pharmaceuticals and biologically active molecules.

30. The biocompatible membrane composite of claim 15, wherein the biocompatible membrane composite has a hydrophilic coating thereon.

31. The biocompatible membrane composite of claim 15, wherein the first layer includes bondable solid features that are bonded to an implantable device or implantable cell system.

32. The biocompatible membrane composite of claim 31, wherein the implantable device comprises, switches, sensors, bolometers, biosensors, chemical sensors, inertial sensors, acoustic sensors, microphones, microspeakers, pressure sensors, resonators, ultrasonic resonators, temperature sensors, vibration sensors, microengines, actuators, thermal actuators, bimorph and unimorph actuators, electrical rotating micromachines, microgears, micropumps, microtransmiitors, microengines, optical micro-electro-mechanical systems, micromirrors, optical switches, and bio-micro-electro-mechanical systems and any combination thereof.

33. (canceled)

34. (canceled)

35. The biocompatible membrane composite of claim 15, wherein the first solid features are at least partially bonded to a cell system or implantable device.

36. The biocompatible membrane composite of claim 35, wherein the cell system is a cell container or a bioactive scaffold.

37. (canceled)

38. (canceled)

39. A method for lowering blood glucose levels in a mammal, the method comprising:
transplanting a cell encapsulation device containing the biocompatible membrane composite of claim 1,
wherein cells encapsulated therein comprise a population of PDX1-positive pancreatic endoderm cells, and
wherein the pancreatic endoderm cells mature into insulin secreting cells, thereby lowering blood glucose.

40.-55. (canceled)

56. A method for producing insulin in vivo, the method comprising:
transplanting a cell encapsulation device containing the biocompatible membrane composite of claim 1 and a population of PDX-1 pancreatic endoderm cells that mature into insulin secreting cells,
wherein the insulin secreting cells secrete insulin in response to glucose stimulation.

57.-63. (canceled)

64. The biocompatible membrane composite of claim 1, wherein the first layer comprises bondable solid features that are bonded to an implantable device or implantable cell system.

65. The biocompatible membrane composite of claim 1, wherein the biocompatible membrane composite has a hydrophilic coating thereon.

66. The biocompatible membrane composite of claim 15, wherein the first biocompatible membrane composite is configured for use in conjunction with tissues, scaffolds, two dimensional cell culture systems, three dimensional cell culture systems, cell containers, cell encapsulation devices, cell systems and combinations thereof.

67. The biocompatible membrane composite of claim 15, wherein at least one of the first layer and the second layer is configured as a bio-interface for implantable sensors that are used to detect molecules produced in the body or molecules that are produced outside the body.

68. The biocompatible membrane composite of claim 15, wherein at least one of the first layer and the second layer is configured as a biocompatible cover for implantable devices that provide or require molecules, signals, or activity within the body to elicit their function.
